(19) United States
(12) Patent Application Publication
(10) Pub. No.: US 2025/0027052 A1
(43) Pub. Date: Jan. 23, 2025

(54) VECTORS INCORPORATING A COMBINATION OF PROMOTERS DRIVING SELECTABLE MARKER EXPRESSION

(71) Applicant: AMGEN INC., Thousand Oaks, CA (US)

(72) Inventors: Eugenia ZAH, Camarillo, CA (US); Natalia GOMEZ, Playa Vista, CA (US); Kristine DARIS, Newbury Park, CA (US); Xing MA, Oak Park, CA (US)

(21) Appl. No.: 18/778,486

(22) Filed: Jul. 19, 2024

Related U.S. Application Data
(60) Provisional application No. 63/528,181, filed on Jul. 21, 2023.

(51) Int. Cl. C12N 5/071; C12N 9/00; C12N 9/06; C12N 15/85

(52) U.S. Cl. CPC C12N 5/0682; C12N 9/003; C12N 9/93; C12N 15/85; C12N 2510/00; C12N 2840/203; C12Y 603/01002

(57) ABSTRACT

Dual vector systems, and mammalian host cells comprising them, for the expression of multi chain molecules are provided where each vector employs a different promoter driving the expression of a selectable marker. The mammalian host cells can be used for producing a recombinant protein of interest in a mammalian cell culture.

### FIELD OF THE INVENTION

The present invention relates generally to mammalian host cells comprising vector systems for the expression of recombinant proteins having three or more chain-containing molecules. In particular, the vector systems contain two expression vectors each expressing a selectable marker where the promoters driving expression of the selectable marker are different.

### BACKGROUND OF THE INVENTION

Due to their broad applications, biologics are used worldwide in a variety of applications, such as therapeutics and diagnostics. Mammalian cell lines are the predominant expression systems for these biologics, with Chinese hamster ovary (CHO) cells being the predominant cellular factory. See Lalonde et al., 2017, J Biotechnol 251:128-140. Particularly with the advent of biosimilars, speed-to-market and cost-efficiency are now more important than ever before.

The costs of manufacturing biologics are high due to their complexity of production utilizing a multistep process involving the selection of optimal cell lines, culturing production cells in large quantities, and purification of the desired biologic from the cell harvest. Manufacturing is typically even more complex for new antibody modalities such as those having three or four antibody chains. While these costs are decreasing due to improvements in all facets of production, their costs can still be prohibitive in their widespread adoption as front-line therapies.

In order to make biological therapeutics more accessible to patients, decreasing the cost of goods for the manufacturing process is an attractive proposition. One way to achieve this objective is to reduce the cost of goods by increasing the titers associated with production cell lines. The right vector configuration can help to optimize expression levels of different chains in recombinant proteins, particularly for three- or four-chain molecules, resulting in more balanced chain expression, reduced impurities and higher product quality.

Attenuation of glutamine synthetase selection marker has been shown to improve product titer. See Sacco et al., 2022, Biotechnol. Bioeng. 119:1712-1727. See also, Yang et al., 2019, Bioprocess Biosyst. Eng. 42:799-806.

There still exists a need for vector systems, which when transfected into host cell lines, produce recombinant proteins, particularly three- and four-chain molecules, at high titers, with ‘minimal impact on growth and productivity. Such vector systems would benefit the process development of biologics.

### SUMMARY OF THE INVENTION

The present disclosure provides a mammalian host cell for expressing an antigen binding protein having three or four different chains comprising a first expression vector and a second expression vector, wherein: (a) the first expression vector comprises nucleotide sequences encoding 1) a first chain and a second chain, wherein the first chain is operably linked to a first promoter, the second chain is operably linked to a second promoter or an IRES sequence or is linked to the first chain through a linker sequence; and 2) a first selectable marker operably linked to a third promoter; and (b) the second expression vector comprises nucleotide sequences encoding 1) a third chain and a fourth chain, wherein the third chain is operably linked to a fourth promoter, the fourth chain is operably linked to a fifth promoter or an IRES sequence or is linked to the third chain through a linker sequence; and 2) a second selectable marker operably linked to a sixth promoter, wherein the first, second, third and fourth chains are selected from a heavy chain, a light chain, an antibody heavy chain fusion, an antibody light chain fusion, an ScFv, an ScFv-Fc, wherein two of the chains may be identical; and wherein the sixth promoter is different from the third promoter.

In certain embodiments, the second chain and the fourth chain are operably linked to a second promoter and fourth promoter, respectively.

In certain embodiments, the first selectable marker and the second selectable marker are the same. In certain aspects, the first selectable marker and the second selectable marker are selected from the group consisting of glutamine synthetase and dihydrofolate reductase. In certain aspects, the first selectable marker and the second selectable marker are glutamine synthetase.

In certain embodiments, the third promoter and the sixth promoter are selected from mPGK and Srα.

In certain embodiments, the first, second, fourth and fifth promoters are different from the third and sixth promoters. In certain aspects, the first, second, fourth and fifth promoters are selected from the group consisting of CMV/GAPDH, CMV/adL, and CMV/EF1α.

In certain embodiments, the first and fourth promoters are identical and the second and fifth promoters are identical. In certain aspects, the first, second, fourth and fifth promoters are identical. In certain aspects, the first and fourth promoters are different from the second and fifth promoters.

In certain embodiments, the stronger promoter of the third promoter and the sixth promoter is on the expression vector with the more difficult-to-express chains. In certain aspects, Srα is on the expression vector with the more difficult-to-express chains and mPGK is on the expression vector with the easier-to-express chains.

In certain aspects of embodiments where the selectable marker is glutamine synthetase, methionine sulfoximine (MSX) stringency is optimized to favor expression of the more difficult-to-express chain paired with the stronger promoter. In certain sub-aspects, the MSX stringency is less than 75 μM.

In certain embodiments, the stronger promoter of the third promoter and the sixth promoter is on the expression vector with the easier-to-express chains.

In certain embodiments, the first and third chains are antibody light chains and the second and fourth chains are antibody heavy chains. In certain aspects, the first and second chains are antibody light chains that are identical. In certain aspects, the first and second chains are antibody light chains that are different.

In certain embodiments, a) the first and third chains are antibody light chains and b) one of the second or fourth chain is an antibody heavy chain and the other is an antibody heavy chain fusion. In certain aspects, the first and second chains are antibody light chains that are identical. In certain aspects, the first and second chains are antibody light chains that are different.

In certain embodiments, wherein the antigen binding protein having three or four chains is selected from the group consisting of heteroIgG or C1mAb.

In certain embodiments, the first expression vector comprises in 5′ to 3′ order a first promoter, a nucleotide sequence encoding a first antibody light chain, a second promoter or an IRES, a nucleotide sequence encoding a first heavy chain, a third promoter which is Srα, and a nucleotide sequence encoding a selectable marker which is glutamine synthetase; and the second expression vector comprises in 5′ to 3′ order a fourth promoter, a nucleotide sequence encoding a second antibody light chain, a fifth promoter or an IRES, a nucleotide sequence encoding a second heavy chain, a sixth promoter which is mPGK, and a nucleotide sequence encoding a selectable marker which is glutamine synthetase. In certain aspects, the first antibody light chain and the second antibody light chain are identical. In certain aspects, the first heavy chain is more difficult-to-express than the second heavy chain.

In certain embodiments, the first expression vector comprises in 5′ to 3′ order a first promoter, a nucleotide sequence encoding a first antibody light chain, a second promoter, a nucleotide sequence encoding a first heavy chain, a third promoter which is Srα, and a nucleotide sequence encoding a selectable marker which is glutamine synthetase; and the second expression vector comprises in 5′ to 3′ order a fourth promoter, a nucleotide sequence encoding a second antibody light chain, a fifth promoter, a nucleotide sequence encoding a second heavy chain, a sixth promoter which is mPGK, and a nucleotide sequence encoding a selectable marker which is glutamine synthetase. In certain aspects, the first antibody light chain and the second antibody light chain are identical. In certain aspects, the first heavy chain is more difficult-to-express than the second heavy chain.

In certain embodiments, the first expression vector comprises in 5′ to 3′ order a first promoter, a nucleotide sequence encoding an antibody light chain, a second promoter or an IRES, a nucleotide sequence encoding a heavy chain fusion, a third promoter which is Srα, and a nucleotide sequence encoding a selectable marker which is glutamine synthetase; and the second expression vector comprises in 5′ to 3′ order a fourth promoter, a nucleotide sequence encoding the antibody light chain, a fifth promoter or an IRES, a nucleotide sequence encoding a heavy chain, a sixth promoter which is mPGK, and a nucleotide sequence encoding a selectable marker which is glutamine synthetase. In certain aspects, the first antibody light chain and the second antibody light chain are identical.

In certain embodiments, the first expression vector comprises in 5′ to 3′ order a first promoter, a nucleotide sequence encoding an antibody light chain, a second promoter, a nucleotide sequence encoding a heavy chain fusion, a third promoter which is Srα, and a selectable marker which is glutamine synthetase; and the second expression vector comprises in 5′ to 3′ order a fourth promoter, a nucleotide sequence encoding the antibody light chain, a fifth promoter, a nucleotide sequence encoding a heavy chain, a sixth promoter which is mPGK, and a nucleotide sequence encoding a selectable marker which is glutamine synthetase. In certain aspects, the first antibody light chain and the second antibody light chain are identical.

In certain embodiments, the first and second expression vectors are integrated in the genome.

In certain embodiments, the host cell is a Chinese Hamster Ovary (CHO) cell. In certain aspects, the CHO cell is deficient in dihydrofolate reductase (DHFR-) or is a glutamine synthetase knock out (GSKO).

The present disclosure also provides a method for producing an antigen binding protein having three or four chains comprising: (a) culturing the mammalian host cell of any of the embodiments, sub-embodiments, aspects, and sub-aspects described above under conditions to express the antigen binding protein; and (b) recovering the antigen binding protein.

The present disclosure also provides a method for producing an antigen binding protein having three or four chains comprising: (a) culturing the mammalian host cell which is a Chinese Hamster Ovary (CHO) cell deficient in dihydrofolate reductase (DHFR-) or is a glutamine synthetase knock out (GSKO) under conditions to express the antigen binding protein and under methotrexate stringency in the case of a CHO DHFR-cell or methionine sulfoxamine in the case of a CHO GSKO cell to favor expression of the difficult-to-express chain paired with the stronger GS promoter; and (b) recovering the antigen binding protein.

In certain embodiments of these methods, the recovered antigen binding protein is purified and formulated in a pharmaceutically acceptable formulation.

The present disclosure also provides an antigen binding protein produced by any of the mammalian host cells provided herein or according to any of the methods provided herein.

### BRIEF DESCRIPTION OF THE DRAWINGS

FIG. 1A-B depicts A) Examples of mixed promoter expression cassettes driving glutamine synthetase (GS) selection for 3- or 4-chain molecules. Promoters for the genes of interest (i.e., the light chain (LC. LC1, and LC2), heavy chain (HC) or heavy chain fusion (HC-mAb)) can be modified depending on molecule of interest. mPGK and SRα refer to the promoters used to drive expression of GS. (B) schematic representation of 3-chain (C1mAb) and 4-chain (heteroIgG) molecules evaluated requiring vector optimization.

FIG. 2A-F shows (A) Schematic of expression cassettes used in vector evaluation study of 3-chain C1mAb molecule. (B) shows mPGK/Srα vector combination yields up to ˜5.3-fold improvement in effective titer normalized to % nrCE main peak. (C) shows reduced % nrCE pre-peak impurities, (D) shows increased % nrCE main peak, (E,F) shows increased relative expression of mAb-fusion as measured by rCE.

FIG. 3A-C shows vectors containing a mixed promoter expression cassettes expressing a heteroIgG improve day 10 effective titers normalized to % SEC main peak up to 1.2-fold (A) with minimal impact on product quality (B,C).

### DETAILED DESCRIPTION OF THE INVENTION

The present disclosure is based in part on the surprising finding that, when two-vector systems are employed in mammalian host cells, the use of different promoters driving expression of the selectable marker can increase the titer of the recombinant protein being produced, particularly for antibody modalities having three- or four-chains. These two-vector systems can be employed in mammalian host cells to produce antibodies having two different heavy chains and two different light chains, where each heavy chain-light-chain pair is expressed from a different vector. These two-vector systems can also be employed for the production of antibody modalities that have three or four unique chains.

Standard antibody production techniques often employ different promoters for the expression of the heavy chain and the light chain to optimize expression of the antibody. This is often necessary because the heavy and light chain are expressed at different levels. The situation becomes even more complicated when the antibody structures contain three or four chains. The inventors have surprisingly found that by employing different promoters for the selectable marker on each vector, the titers of the resulting antibody structures can be increased with improved product quality (e.g., reduced aggregation (high molecular weight impurities (HMW) and reduced degradation (low molecular weight impurities (LMW)).

By employing the two-vector systems and mammalian host cells described herein, production of recombinant proteins can be increased while retaining or improving product quality. By using the two-vector systems described herein in mammalian host production cell lines, biopharmaceuticals can be produced in a less expensive and more consistent manner. The invention finds particular utility in the commercial production of antibodies having three or four unique chains.

The vector systems described herein are employed in cell lines (also referred to as “host cells”), preferably mammalian (“mammalian host cells”), grown in cell culture media to produce a recombinant protein of commercial or scientific interest. Cell lines are typically derived from a lineage arising from a primary culture that can be maintained in culture for an unlimited time. Genetically engineering the cell line involves transfecting, transforming or transducing the cells with the two-vector system where each vector contains nucleotide sequences encoding two antibody chains so as to cause the host cell to express an antigen binding protein of interest. Methods and vectors for genetically engineering cells and/or cell lines to express, for example, a protein of interest, are well known to those of skill in the art; for example, various techniques are illustrated in Current Protocols in Molecular Biology, Ausubel et al., eds. (Wiley & Sons, New York, 1988, and quarterly updates); Sambrook et al., Molecular Cloning: A Laboratory Manual (Cold Spring Laboratory Press, 1989); Kaufman, R. J., Large Scale Mammalian Cell Culture, 1990, pp. 15-69; and Harlow and Lane Antibodies: A Laboratory Manual Cold Spring Harbor Laboratory Press, Cold Spring Harbor, N.Y. (1990).

### Definitions

While the terminology used in this application is standard within the art, definitions of certain terms are provided herein to assure clarity and definiteness in the meaning of the claims. Units, prefixes, and symbols may be denoted in their SI (International System of Units) accepted form. Numeric ranges recited herein are inclusive of the numbers defining the range and include and are supportive of each integer within the defined range. The methods and techniques described herein are generally performed according to conventional methods well known in the art and as described in various general and more specific references that are cited and discussed throughout the present specification unless otherwise indicated.

As used herein, the terms “a” and “an” mean one or more unless specifically indicated otherwise. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well-known and commonly used in the art.

All documents, or portions of documents, cited in this application, including but not limited to patents, patent applications, articles, books, and treatises, are hereby expressly incorporated by reference. What is described in an embodiment of the invention can be combined with other embodiments of the invention.

The present disclosure provides methods of expressing a “protein of interest”. A “protein of interest” includes naturally occurring proteins, recombinant proteins, and engineered proteins (e.g., proteins that do not occur in nature and which have been designed and/or created by humans). A protein of interest can, but need not be, a protein that is known or suspected to be therapeutically relevant.

As used herein, an “antibody chain” or “chain” refers to antibody light chains, antibody heavy chains, antibody light chain fusions, antibody heavy chain fusions, scFv-Fc fusion, VHH fusions, and the like. The terms “antibody heavy chain” and “antibody light chain” have the standard meaning in the art and include, for example, the various antibody heavy and light chains described elsewhere herein (e.g. heavy and light chains of lgG1, lgG2, lgG3, and lgG4 mAbs). The terms “antibody heavy chain” and “antibody light chain” include standard full-length heavy chains and light chains. The terms “antibody heavy chain fusion”. “antibody heavy chain fusion protein”, “heavy chain-protein fusion”, and “heavy chain-peptide fusion” are used interchangeably herein and refer to a polypeptide that contains an antibody heavy chain that is covalently linked to one or more additional proteins or peptides. For example, an “antibody heavy chain fusion” can be an antibody heavy chain covalently linked to a cytokine, VHH or an scFv. The linkage may be direct, or via a peptide linker (e.g. a glycine-serine linker). In an antibody heavy chain fusion, the antibody heavy chain may be linked to additional protein(s) at the N-terminus or the C-terminus of the heavy chain (or both locations). The terms “antibody light chain fusion protein” and “antibody light chain fusion” have the same meaning as described immediately above for “antibody heavy chain fusion”, except for an antibody light chain replacing the antibody heavy chain. As used herein, an “antibody fusion” refers to an antibody as provided herein which is covalently linked to one or more additional proteins or polypeptides (e.g. via a heavy chain or light chain of the antibody). Thus, an antibody fusion contains at least an antibody heavy chain fusion or an antibody light chain fusion as one of the chains of the antibody fusion. Most commonly, an antibody fusion is a molecule that contains two antibody light chains, one antibody heavy chain, and one antibody heavy chain fusion, such that the additional protein is linked to one of the heavy chains of the antibody. One example is C1mAb which has a fusion at the C-terminus of the Fc region.

As used herein, the terms “polypeptide” and “protein” (e.g., as used in the context of a protein of interest or a polypeptide of interest) are used interchangeably herein to refer to a polymer of amino acid residues. The terms also apply to amino acid polymers in which one or more amino acid residues is an analog or mimetic of a corresponding naturally occurring amino acid, as well as to naturally occurring amino acid polymers. The terms can also encompass amino acid polymers that have been modified, e.g., by the addition of carbohydrate residues to form glycoproteins, or phosphorylated. Polypeptides and proteins can be produced by a naturally-occurring and non-recombinant cell, or polypeptides and proteins can be produced by a genetically-engineered or recombinant cell. Polypeptides and proteins can comprise molecules having the amino acid sequence of a native protein, or molecules having deletions from, additions to, and/or substitutions of one or more amino acids of the native sequence.

As used herein, the term “heterologous” used in connection with a nucleic acid means having a nucleic acid not naturally occurring within a host cell. This can include mutated sequences, e.g., sequences differing from the naturally occurring sequence. This can include sequences from other species. This can also include having a sequence at a different position in the genome than that naturally-occurring in the host cell. This generally does not include natural mutations that may occur in a host cell. A cell already containing a heterologous nucleic acid encoding a protein of interest, for example, by stable integration of an expression cassette, would be considered to contain a heterologous nucleic acid sequence. For clarity, a CHO cell or a derivative thereof (e.g., a DHFR- or GS knockout) having a nucleic acid encoding an antigen binding protein would be considered to have a heterologous nucleic acid.

As used herein, the term “operably linked” refers to that the nucleic acid sequences being linked are typically contiguous, or substantially contiguous, and, where necessary to join two protein coding regions, contiguous and in reading frame. However, since enhancers generally function when separated from the promoter by several kilobases and intronic sequences may be of variable lengths, some polynucleotide elements may be operably linked but not contiguous.

As used herein, the term “bioreactor” means any vessel useful for the growth of a cell culture. The cell cultures of the mammalian cells described herein can be grown in a bioreactor, which can be selected based on the application of a protein of interest that is produced by cells growing in the bioreactor. A bioreactor can be of any size so long as it is useful for the culturing of cells; typically, a bioreactor is sized appropriate to the volume of cell culture being grown inside of it. Typically, a bioreactor will be at least 1 liter and may be 2, 5, 10, 50, 100, 200, 250, 500, 1,000, 1500, 2000, 2,500, 5,000, 8,000, 10,000, 12,000 liters or more, or any volume in between. The internal conditions of the bioreactor, including, but not limited to pH and temperature, can be controlled during the culturing period. Those of ordinary skill in the art will be aware of, and will be able to select, suitable bioreactors for use in practicing the methods disclosed herein based on the relevant considerations.

As used herein, “cell culture” or “culture” is meant the growth and propagation of cells outside of a multicellular organism or tissue. Suitable culture conditions for mammalian cells are known in the art. See e.g. Animal cell culture: A Practical Approach, D. Rickwood, ed., Oxford University Press, New York (1992). Mammalian cells may be cultured in suspension or while attached to a solid substrate. Fluidized bed bioreactors, hollow fiber bioreactors, roller bottles, shake flasks, or stirred tank bioreactors, with or without microcarriers, can be used. In one embodiment 500 L to 2000 L bioreactors are used. In one embodiment, 1000 L to 2000 L bioreactors are used.

The term “cell culture medium” (also called “culture medium,” “cell culture media,” “tissue culture media,”) refers to any nutrient solution used for growing cells, e.g., animal or mammalian cells, and which generally provides at least one or more components from the following: an energy source (usually in the form of a carbohydrate such as glucose); one or more of all essential amino acids, and generally the twenty basic amino acids, plus cysteine; vitamins and/or other organic compounds typically required at low concentrations; lipids or free fatty acids; and trace elements, e.g., inorganic compounds or naturally occurring elements that are typically required at very low concentrations, usually in the micromolar range.

The nutrient solution may optionally be supplemented with additional optional components to optimize growth of cells, such as hormones and other growth factors, e.g., transferrin, epidermal growth factor, serum, and the like; salts, e.g., calcium, magnesium and phosphate, and buffers, e.g., HEPES; nucleosides and bases, e.g., adenosine, thymidine, hypoxanthine; and protein and tissue hydrolysates, e.g., hydrolyzed animal or plant protein (peptone or peptone mixtures, which can be obtained from animal byproducts, purified gelatin or plant material); antibiotics, e.g., gentamycin; anti-clumping agents; cell protectants or surfactants such as Pluronic®F68 (also referred to as Lutrol® F68 and Kolliphor® P188; nonionic triblock composed of a central hydrophobic chain of polyoxypropylene (poly(propylene oxide)) flanked by two hydrophilic chains of polyoxyethylene (poly(ethylene oxide)); polyamines, e.g., putrescine, spermidine and spermine (see e.g., International Patent Application Publication No. WO 2008/154014) and pyruvate (see e.g. U.S. Pat. No. 8,053,238) depending on the requirements of the cells to be cultured and/or the desired cell culture parameters.

Cell culture media include those that are typically employed in and/or are known for use with any cell culture process, such as, but not limited to, batch, extended batch, fed-batch and/or perfusion or continuous culturing of cells.

A “base” (or batch) cell culture medium refers to a cell culture medium that is typically used to initiate a cell culture and is sufficiently complete to support the cell culture.

A “fed-batch culture” refers to a form of suspension culture and means a method of culturing cells in which additional components are provided to the culture at a time or times subsequent to the beginning of the culture process. The provided components typically comprise nutritional supplements for the cells which have been depleted during the culturing process. Additionally or alternatively, the additional components may include supplementary components (e.g., a cell-cycle inhibitory compound). A fed-batch culture is typically stopped at some point and the cells and/or components in the medium are harvested and optionally purified.

A “growth” cell culture medium refers to a cell culture medium that is typically used in cell cultures during a period of exponential growth, a “growth phase”, and is sufficiently complete to support the cell culture during this phase. A growth cell culture medium may also contain selection agents that confer resistance or survival to selectable markers incorporated into the host cell line. Such selection agents include, but are not limited to, geneticin (G418), neomycin, hygromycin B, puromycin, zeocin, methionine sulfoaimine, methotrexate, glutamine-free cell culture medium, cell culture medium lacking glycine, hypoxanthine and thymidine, or thymidine alone.

A “perfusion” cell culture medium refers to a cell culture medium that is typically used in cell cultures that are maintained by perfusion or continuous culture methods and is sufficiently complete to support the cell culture during this process. Perfusion cell culture medium formulations may be richer or more concentrated than base cell culture medium formulations to accommodate the method used to remove the spent medium. Perfusion cell culture medium can be used during both the growth and production phases.

A “production” cell culture medium refers to a cell culture medium that is typically used in cell cultures during the transition when exponential growth is ending and protein production takes over, “transition” and/or “product” phases, and is sufficiently complete to maintain a desired cell density, viability and/or product titer during this phase.

Concentrated cell culture medium can contain some or all of the nutrients necessary to maintain the cell culture; in particular, concentrated medium can contain nutrients identified as or known to be consumed during the course of the production phase of the cell culture. Concentrated medium may be based on just about any cell culture media formulation. Such a concentrated feed medium can contain some or all the components of the cell culture medium at, for example, about 2×, 3×, 4×, 5×, 6×, 7×, 8×, 9×, 10×, 12×, 14×, 16×, 20×, 30×, 50×, 100×, 200×, 400×, 600×, 800×, or even about 1000× of their normal amount.

The components used to prepare cell culture medium may be completely milled into a powder medium formulation; partially milled with liquid supplements added to the cell culture medium as needed; or added in a completely liquid form to the cell culture.

Cell cultures can also be supplemented with independent concentrated feeds of particular nutrients which may be difficult to formulate or are quickly depleted in cell cultures. Such nutrients may be amino acids such as tyrosine, cysteine and/or cystine (see e.g., International Patent Application Publication No. WO2012/145682). For example, a concentrated solution of tyrosine can independently be fed to a cell culture grown in a cell culture medium containing tyrosine, such that the concentration of tyrosine in the cell culture does not exceed 8 mM. In another example, a concentrated solution of tyrosine and cystine is independently fed to the cell culture being grown in a cell culture medium lacking tyrosine, cystine or cysteine. The independent feeds can begin prior to or at the start of the production phase. The independent feeds can be accomplished by fed batch to the cell culture medium on the same or different days as the concentrated feed medium. The independent feeds can also be perfused on the same or different days as the perfused medium.

“Serum-free” applies to a cell culture medium that does not contain animal sera, such as fetal bovine serum. Various tissue culture media, including defined culture media, are commercially available, for example, any one or a combination of the following cell culture media can be used: RPMI-1640 Medium, RPMI-1641 Medium, Dulbecco's Modified Eagle's Medium (DMEM), Minimum Essential Medium Eagle, F-12K Medium, Ham's F12 Medium, Iscove's Modified Dulbecco's Medium, McCoy's 5A Medium, Leibovitz's L-15 Medium, and serum-free media such as EX-CELL™ 300 Series (JRH Biosciences, Lenexa, Kansas), MCDB 302 (Sigma Aldrich Corp., St. Louis, MO), among others. Serum-free versions of such culture media are also available. Cell culture media may be supplemented with additional or increased concentrations of components such as amino acids, salts, sugars, vitamins, hormones, growth factors, buffers, antibiotics, lipids, trace elements and the like, depending on the requirements of the cells to be cultured and/or the desired cell culture parameters. Customized cell culture media can also be used.

“Titer” means the total amount of a polypeptide or protein of interest (which may be a naturally occurring or recombinant protein of interest) produced by a cell culture in a given amount of medium volume. Titer can be expressed in units of milligrams or micrograms of polypeptide or protein per milliliter (or other measure of volume) of medium. “Cumulative titer” is the titer produced by the cells during the course of the culture, and can be determined, for example, by measuring daily titers and using those values to calculate the cumulative titer.

As used herein, the term “host cell” is understood to include a cell that has been genetically engineered to express a polypeptide of interest. Genetically engineering a cell involves transfecting, transforming or transducing the cell with a nucleic acid encoding a recombinant polynucleotide molecule (a “gene of interest”), and/or otherwise altering (e.g., by homologous recombination and gene activation or fusion of a recombinant cell with a non-recombinant cell) so as to cause the host cell to express a desired recombinant polypeptide. Methods and vectors for genetically engineering cells and/or cell lines to express a polypeptide of interest are well known to those of skill in the art; for example, various techniques are illustrated in Current Protocols in Molecular Biology. Ausubel et al., eds. (Wiley & Sons, New York, 1988, and quarterly updates); Sambrook et al., Molecular Cloning: A Laboratory Manual (Cold Spring Laboratory Press, 1989); Kaufman, R. J., Large Scale Mammalian Cell Culture, 1990, pp. 15-69. The term includes the progeny of the parent cell, whether or not the progeny is identical in morphology or in genetic makeup to the original parent cell, so long as the gene of interest is present. A cell culture can comprise one or more host cells.

It is understood that wherever embodiments are described herein with the language “comprising,” otherwise analogous embodiments described in terms of “consisting of” and/or “consisting essentially of” are also provided.

### Selectable Markers

For stable transfection of mammalian cells, it is known that, depending upon the expression vector and transfection technique used, only a small fraction of cells may integrate the foreign DNA into their genome. In order to identify and select these integrants, a gene that encodes a selectable marker is generally introduced into the host cells in the same expression vector as the gene(s) of interest.

A selectable marker gene encodes a protein necessary for the survival and growth of a host cell grown in a selective culture medium. Typical selection marker genes encode proteins that (a) confer resistance to antibiotics or other toxins, e.g., ampicillin, tetracycline, or kanamycin for prokaryotic host cells; (b) complement auxotrophic deficiencies of the cell; or (c) supply critical nutrients not available from complex or defined media through metabolism. Specific antibiotic-resistance selectable markers are the kanamycin resistance gene, the ampicillin resistance gene, the tetracycline resistance gene, and neomycin resistance gene.

Other selectable genes may be used to amplify the gene that will be expressed. Amplification is the process wherein genes that are required for production of a protein critical for growth or cell survival are reiterated in tandem within the chromosomes of successive generations of recombinant cells. Examples of suitable selectable markers for mammalian cells include glutamine synthetase (GS), dihydrofolate reductase (DHFR), asparaginase (Aspg; see Ha et al. Biotechnol Bioeng. 2023 120:1159-1166) and promoterless thymidine kinase genes.

Mammalian cell transformants are placed under selection pressure wherein only the transformants are uniquely adapted to survive by virtue of the selectable gene present in the vector. Selection pressure is imposed by culturing the transformed cells under conditions in which the concentration of selection agent in the medium is successively increased, thereby leading to additional stringency and/or amplification of both the selectable gene and the DNA that encodes a protein of interest. As a result, increased quantities of a polypeptide of interest are synthesized from the amplified DNA. The selection agent for GS is methionine sulfoxamine (MSX). The selection agent for DHFR is methotrexate (MTX).

Relative to DHFR-based system, GS knockout cell lines (GSKO) provide sufficient selection stringency without MSX or with low MSX concentrations, while 25 μM MSX coupled with the GS-knockout cell line led to higher selection efficiency compared with CHOK1SV cell lines at higher MSX concentrations (Fan, et al., Biotechnol Bioeng., 109 (4): 1007-1015 (2012)). A previous report showed that increasing the MSX concentration in the seed train stage after clone selection increased productivity without significant impacts on cell growth, GS and target gene copy numbers and expression, and maintained product quality attributes in multiple GS knockout cell lines (Tian et al., Engineering in Life Sciences 20 (3-4): 112-125 (2020)). Chain/vector expression can be influenced by increasing stringency during pool recovery/selection by adding MSX. MSX can also be used to favor different vectors in the mixed GS promoter case.

In certain embodiments, the MSX concentration can be optimized for one of the promoters driving GS expression. In a specific embodiment, the MSX concentration is optimized for the GS linked to the more difficult to express chain.

In certain embodiments, the selectable marker is glutamine synthetase. Glutamine synthetase (GS) catalyzes glutamine biosynthesis by the condensation of ammonia with glutamate. Mammalian GS enzyme is a decamer composed of two stacked pentameric rings, with ten active sites located at the junction of subunits. Each active site is formed by residues from the N-terminal domain (the β-grasp domain, composed of residues 25-112) of one subunit and by residues from the C-terminal domain (the catalytic domain, composed of residues 113-373) of an adjacent subunit.

In certain embodiments, a CHO DHFR-cell or a CHO GSKO cell can be cultured under conditions to express the antibody chains under methotrexate stringency in the case of a CHO DHFR-cell or methionine sulfoxamine stringency in the case of a CHO GSKO cell to favor expression of the difficult-to-express chain paired with the stronger GS promoter.

In certain embodiments, a CHO DHFR-cell or a CHO GSKO cell can be cultured under conditions to express the antibody chains under methotrexate stringency in the case of a CHO DHFR-cell or methionine sulfoxamine stringency in the case of a CHO GSKO cell to favor expression of the difficult-to-express chain paired with the weaker GS promoter.

In one embodiment the MSX stringency is less than 75 μM. In one embodiment the stringency is less than 50 μM. In one embodiment the stringency is about 25 μM to 50 μM. In one embodiment the stringency is about 25 μM to 45 μM. In one embodiment the stringency is about 25 μM to 40 M. In one embodiment the stringency is about 25 μM to 35 μM. In one embodiment the stringency is about 25 μM to 30 μM. In one embodiment the stringency is less than or equal to 25 μM. In one embodiment the stringency is about 5 μM to 25 μM. In one embodiment the stringency is about 10 μM to 25 μM. In one embodiment the stringency is about 15 μM to 25 μM. In one embodiment the stringency is about 20 μM to 25 μM. In one embodiment the stringency is 5, 10, 11, 12, 13, 14, 15, 16, 17, 18, 19, 20, 21, 22, 23, 24, 25, 26, 27, 28, 29, 30, 35, 40, 45, 50, 55, 60, 65, 70, or 75 μM.

### Mixed Promoter

Expression and cloning will typically contain a promoter that is recognized by the host organism and operably linked to the nucleotide sequence encoding a protein of interest. Promoters are untranscribed sequences located upstream (i.e., 5′) to the start codon of a structural gene (generally within about 100 to 1000 bp) that control transcription of the structural gene. Promoters are conventionally grouped into one of two classes: inducible promoters and constitutive promoters. Inducible promoters initiate increased levels of transcription from DNA under their control in response to some change in culture conditions, such as the presence or absence of a nutrient or a change in temperature. Constitutive promoters, on the other hand, uniformly transcribe a gene to which they are operably linked, that is, with little or no control over gene expression. A large number of promoters, recognized by a variety of potential host cells, are well known.

In the two-vector systems described herein, any suitable combination of promoters can be used so long as the two promoters are different, appropriate for the host cell line and drive expression to a linked gene to a different degree. Chain ratios of the expressed polypeptides can be measured using techniques well-known in the art. Such ratios can provide information on the more difficult-to-express chain and the easier-to-express chain. Chain ratios of the expressed polypeptides can be measured using techniques well-known in the art, such as reduced capillary electrophoresis-sodium dodecyl sulfate (rCE-SDS). Suitable promoters for use with mammalian host cells are well known and include, but are not limited to, those obtained from the genomes of viruses such as polyoma virus, fowlpox virus, adenovirus (such as Adenovirus 2), bovine papilloma virus, avian sarcoma virus, cytomegalovirus, retroviruses, hepatitis-B virus, and Simian Virus 40 (SV40). Other suitable mammalian promoters include heterologous mammalian promoters, for example, heat-shock promoters and the actin promoter.

Additional promoters which may be of interest include, but are not limited to: SV40 early promoter (Benoist and Chambon, 1981, Nature 290:304-310); CMV promoter (Thornsen et al., 1984, Proc. Natl. Acad. U.S.A. 81:659-663); the promoter contained in the 3′ long terminal repeat of Rous sarcoma virus (Yamamoto et al., 1980, Cell 22:787-797); herpes thymidine kinase promoter (Wagner et al., 1981, Proc. Natl. Acad. Sci. U.S.A. 78:1444-1445); promoter and regulatory sequences from the metallothionine gene (Prinster et al., 1982, Nature 296:39-42); and prokaryotic promoters such as the beta-lactamase promoter (Villa-Kamaroff et al., 1978, Proc. Natl. Acad. Sci. U.S.A. 75:3727-3731); the tac promoter (DeBoer et al., 1983, Proc. Natl. Acad. Sci. U.S.A. 80:21-25), the human elongation factor 1α promoter (EF-1α) (Kim et al., 1990, Gene 91:217-233), the SR alpha promoter (Srα) (Takebe et al., 1988, Mol Cell. Biol. January; 8 (1): 466-72, and the mouse mPGK-1 promoter (mPGK) (Adra et al., 1987, Gene 60:65-74).

Promoters of particular interest for nucleotide sequences encoding antibody chains include the Human cytomegalovirus IE1 gene promoter-enhancer (CMV) (Boshart et al., 1985, Cell 41:521-30, GenBank Accession No. X03922), Adenovirus tripartite leader (adl) (Gingeras et al., 1982, J. Biol. Chem. 257:13475-91, GenBank Accession No. J01917), and Hamster glyceraldehyde-3-phosphate dehydrogenase promoter and intron (GAPDH) (U.S. Pat. No. 10,202,261).

In certain embodiments, the promoters for the antibody chains are selected from the combinations of CMV/GAPDH, CMV/adL, and CMV/EF1α. In these combinations of promoters, both the CMV promoter and the promoter selected from GAPDH, adL and EF1a are operably linked to the nucleotide sequence encoding the antibody chain, typically such that the combination is a better promoter than the individual promoters. In certain embodiments, the CMV promoter is 5′ of the promoter selected from GAPDH, adL and EF1α.

In certain embodiments, the promoters for the selectable marker are selected from mPGK and Srα.

Promoters in CHO cells have also been explored. See, e.g., Tossolini et al., 2022, Plasmid 119-120:102620; Nguyen et al., 2019, Biotechnology. J. 14:e1900125; Romanova et al., 2018, Biotechnology. J. 13:e1700232.

In the vector systems disclosed herein, two different promoters each drive the expression of a selectable marker on the two vectors. The promoters are of different strength. Promoter strength comparisons can be found in the literature. See, e.g., Qin et al., 2010, PloS One 5:e10611. Promoter strength can be predicted based on its sequence and comparison to a promoter library (See, e.g., Zhang et Al., 2022, ACS Synth Biol. 11:92-102) or various web tools (See Skrlj et Al., 2010, Anal Biochem. 396:83-90; Liang et al., 2021, Anal. Biochem. 630:114335). Srα is known to be a stronger promoter than the mPGK promoter.

Surprisingly, it has been found that using two different promoters with the selectable marker results in higher production of the antibody regardless of which chains are being produced. However, it has been found that the highest production is seen when the stronger promoter is on the same vector as the more difficult-to-express chains. Accordingly, in certain embodiments, the stronger promoter is on the vector with the difficult-to-express chains. In other embodiments, the stronger promoter is on the vector with the easier-to-express chains.

Ease of expression of antibody chains can be assessed in systems where the vectors are identical except for the chains being produced. Transfecting the vectors into the identical cell line, whether in the same cells or a different pool of cells under the same conditions. As described above, measuring chain ratios can inform on which chains are more difficult-to-express and which chains are easier-to-express.

In certain embodiments, one vector contains Srα operably linked to the selectable marker and the other vector contains mPGK operably linked to the selectable marker. In one aspect of this embodiment, the selectable marker is GS. In one aspect, the vector that contains Srα is on the vector that contains genes encoding chains that are more difficult-to-express, while mPGK is on the vector containing genes encoding chains that are easier-to-express. In another aspect, the vector that contains Srα is on the vector that contains genes encoding chains that are easier to express, while mPGK is on the vector containing genes encoding chains that are more difficult to express.

### 3- and 4-Chain Molecules

A typical antibody is a Y-shaped molecule having four polypeptide chains-two identical heavy chains and two identical light chains. Such an antibody is preferably expressed from a single vector. However, bispecific antibodies require the use of alternative formats and are commonly expressed on two different vectors. See, e.g., Spiess et al., 2015, Mol. Immunol. 67:95-106; Brinkmann et al., 2017, MAbs 9:192-212; and Ma et al., 2021, Frontiers in Immunology 12:626616.

Any format having at least three different chains or that can be expressed on two vectors can be used with the vector systems of the invention. Certain embodiments are described below. Other embodiments are described in the cited references. Others can be determined by those skilled in the art.

An expression vector provided herein contains at least two antibody chain-encoding genes. The two-antibody chain-encoding genes may be the same or different genes. For example, in some embodiments, a vector or nucleic acid construct may contain two copies of the same antibody light chain gene. Alternatively, in some embodiments, a vector or nucleic acid construct may contain one copy of an antibody light chain gene and one copy of an antibody heavy chain gene, or one copy of an antibody light chain gene from a first antibody and one copy of an antibody light chain gene from a second antibody. Alternatively, in some embodiments, a first expression vector may contain one copy of a first antibody light chain and one copy of a first antibody heavy chain. The second expression may contain one copy of a second antibody light chain and one copy of a second antibody heavy chain. In some embodiments, both the first and second expression vectors contain the same antibody light chain sequence and the first and second expression contain different antibody heavy chain sequences. In these embodiments, the two heavy chains may connect through a knob-in-hole or similar mechanism.

In certain embodiments, the two expression vectors, in a mammalian host cell, together produce a bispecific antibody. Bispecific antibodies can be divided into five categories (one of which will not be discussed further as it requires chemical conjugation). See Spiess, et al., 2015, Mol. Immunol. 67:95-106.

Bispecific IgG (BsIgG) is a format that is monovalent for each antigen and produced using two different heavy chains and two different light chains. A Bs IgG can also referred to as a heteroIgG. In this format, typically, the coding sequences for a heavy and light chain pair are on a first vector and the coding sequences for a different heavy and light chain pair are on a second vector.

Appended IgG is a format where additional antigen-binding units are added to either the amino or carboxy termini of either the light or heavy chain. Examples of appended IgG include single domain antibodies (unpaired V_{L} or V_{H}) such as a dual variable domain Ig (DVD-Ig), paired antibody variable domains (e.g., Fv or ScFv), engineered protein scaffolds. See also LaFleur et al., 2013, mAbs 5:208-218; Wu et al., 2007, Nat. Biotechnol. 25:1290-7. In this format, typically, the coding sequences for the heavy and light chain pair are present on both the first and second vector. A coding sequence for an antigen binding unit, e.g., an scFv, a VHH domain or a cytokine, can be added to the coding sequence for either heavy chain or the coding sequence for either light chain. More than one coding sequence for an antigen binding unit can be added.

Bispecific antibody fragments lack antibody constant domains and can be composed of heavy and light chains connected by peptide linkers, scFv fragments, dual-affinity re-targeting proteins (DARTs), diabodies including tetravalent tandem diabodies. See also Arndt et al., 1999, Blood 94:2562-2568; and Kipriyanov et al., 1999, J. Mol. Bio. 293:41-56. In this format, as examples, two different ScFv fragments can be expressed on each of two vectors or, in the case of a DART, one vector contains coding sequence for a VHA-linker-VLB and the other vector contains coding sequence for a VHB-linker-VLA.

Bispecific fusion proteins are antibody fragments linked to other proteins such as receptors and albumin. In this format, typically, the coding sequences for the heavy and light chain pair are present in the first and second vector. A coding sequence for an antigen binding unit, e.g., a receptor, can be added to either the coding sequence for either heavy chain or the coding sequence for either light chain. More than one coding sequence for an antigen binding unit can be added.

In another example, for expressing bispecific IgG antibodies, it may be desirable to use embodiments in which the first gene encoding the antibody light chain and the second gene encoding the antibody light chain encode different antibody light chains, and the first gene encoding the antibody heavy chain and the second gene encoding the antibody heavy chain encode the different antibody heavy chains. This is useful because typical bispecific IgG antibodies contain four different polypeptide types: i) antibody light chain for the first antigen binding portion, ii) antibody heavy chain for the first antigen binding portion, iii) antibody light chain for the second antigen binding portion, and iv) antibody heavy chain for the second antigen binding portion. Accordingly, for bispecific IgG expression it may be desirable to have the first position in the first vector containing a gene encoding a first antibody light chain, the second position in the first vector containing a gene encoding a second antibody light chain, the first position in the second vector containing a gene encoding a first antibody heavy chain, and the second position in the second vector containing a gene encoding a second antibody heavy chain. Alternatively, for bispecific IgG expression it may be desirable to have the first position in the first vector containing a gene encoding a first antibody light chain, the second position in the first vector containing a gene encoding a first antibody heavy chain, the first position in the second vector containing a gene encoding a second antibody light chain, and the second position in the second vector containing a gene encoding a second antibody heavy chain. As discussed above, additional sequences can be appended to the amino- or carboxy-termini of either light chain or either heavy chain.

Other related embodiments are also provided herein. For example, in some embodiments a first vector and second vector may be used with embodiments provided here, such as for the expression of a bispecific antibody having a common light chain (see, e.g. WO2021/124073). A bispecific antibody having a common light chain may contain two different heavy chains.

In embodiments provided herein involving a first vector and a second vector, in some other aspects, the first vector contains a first gene encoding an antibody light chain and a second gene encoding an antibody light chain, and the second vector contains a first gene encoding an antibody heavy chain fusion and a second gene encoding an antibody heavy chain. In some embodiments, the first gene encoding the antibody light chain and the second gene encoding the antibody light chain encode the same antibody light chain (e.g. two copies of the same gene). In some other embodiments, the first gene encoding the antibody light chain and the second gene encoding the antibody light chain encode different antibody light chains (e.g. one copy each of two different antibody light chain genes).

In embodiments provided herein involving a first vector and a second vector, in some other aspects, the first vector contains a first gene encoding an antibody light chain and a second gene encoding an antibody heavy chain, and the second vector contains a first gene encoding an antibody light chain and a second gene encoding an antibody heavy chain. In some embodiments, the first gene encoding the antibody light chain and the second gene encoding the antibody light chain encode the same antibody light chain (e.g. two copies of the same gene). In some other embodiments, the first gene encoding the antibody light chain and the second gene encoding the antibody light chain encode different antibody light chains (e.g. one copy each of two different antibody light chain genes).

The choice of whether to have two copies of the same antibody light chain or one copy each of two different antibody light chains in the first vector depends on specific type of antibody molecule to be manufactured. For example, for an antibody fusion protein involving a standard monospecific IgG mAb covalently linked to another protein via the antibody heavy chain, the molecule typically contains 3 separate polypeptide types: i) antibody light chain; ii) antibody heavy chain; and iii) antibody heavy chain fusion. In this antibody fusion molecule there is one chain each of the antibody heavy chain and antibody heavy chain fusion, and two copies of the antibody light chain. Since there is only one type of antibody light chain in this molecule, two copies of the same antibody light chain gene are provided in the first vector. In another example, for an antibody fusion protein involving a bispecific IgG mAb covalently linked to another protein via the antibody heavy chain, the molecule typically contains 4 separate polypeptide types: i) antibody light chain for the first antigen binding portion; ii) antibody heavy chain for the first antigen binding portion; iii) antibody light chain for the second antigen binding portion; and iv) antibody heavy chain fusion, in which the antibody heavy chain portion is for the second antigen binding portion. In this antibody fusion protein molecule there is one copy of each of the antibody heavy chain and antibody heavy chain fusion, and one copy of each of the antibody light chains. Since there are two types of antibody light chain in this molecule, two different antibody light chain genes are provided in the first vector.

In embodiments provided herein involving two different antibody heavy chains or an antibody heavy chain and an antibody heavy chain fusion, optionally, the heavy chains may contain one or more amino acid modifications in the constant region of the heavy chains to promote heterodimer formation between the two different heavy chains. Such modifications are known in the art and include, for example, charged-based electrostatic amino acid modifications, and steric-based “knob into hole” amino acid modifications.

Vectors are useful for transformation of a host cell and can contain additional nucleic acid sequences that direct and/or control (in conjunction with the host cell) expression of one or more heterologous coding regions operatively linked thereto. An expression construct may include, but is not limited to, sequences that affect or control transcription, translation, and, if introns are present, affect RNA splicing of a coding region operably linked thereto. “Operably linked” means that the components to which the term is applied are in a relationship that allows them to carry out their inherent functions. For example, a control sequence, e.g., a promoter, in a vector that is “operably linked” to a protein coding sequence are arranged such that normal activity of the control sequence leads to transcription of the protein coding sequence resulting in recombinant expression of the encoded protein.

Vectors may be selected to be functional in the particular host cell employed (i.e., the vector is compatible with the host cell machinery, permitting amplification and/or expression of the gene can occur). In some embodiments, vectors are used that employ protein-fragment complementation assays using protein reporters, such as dihydrofolate reductase (see, for example, U.S. Pat. No. 6,270,964). Suitable expression vectors are known in the art and are also commercially available.

Typically, vectors used in host cells will contain sequences for plasmid maintenance and for cloning and expression of exogenous nucleotide sequences. Such sequences will typically include one or more of the following nucleotide sequences (in addition to the promoter(s) and selectable marker described above): one or more enhancer sequences, an origin of replication, transcriptional and translational control sequences, a transcriptional termination sequence, a complete intron sequence containing a donor and acceptor splice site, various pre- or pro-sequences to improve glycosylation or yield, a native or heterologous signal sequence (leader sequence or signal peptide) for polypeptide secretion, a ribosome binding site, a polyadenylation sequence, internal ribosome entry site (IRES) sequences, an expression augmenting sequence element (EASE), tripartite leader (TPA) and VA gene RNAs from Adenovirus 2, and a polylinker region for inserting the polynucleotide encoding the polypeptide to be expressed. Vectors may be constructed from a starting vector such as a commercially available vector, additional elements may be individually obtained and ligated into the vector. Methods used for obtaining each of the components are well known to one skilled in the art.

Vector components may be homologous (i.e., from the same species and/or strain as the host cell), heterologous (e.g., from a species other than the host cell species or strain), hybrid (i.e., a combination of flanking sequences from more than one source), synthetic or native. The sequences of components useful in the vectors may be obtained by methods well known in the art, such as those previously identified by mapping and/or by restriction endonuclease. In addition, they can be obtained by polymerase chain reaction (PCR) and/or by screening a genomic library with suitable probes.

A ribosome-binding site is usually necessary for translation initiation of mRNA and is characterized by a Shine-Dalgarno sequence (prokaryotes) or a Kozak sequence (eukaryotes). The element is typically located 3′ to the promoter and 5′ to the coding sequence of the polypeptide to be expressed.

An origin of replication aids in the amplification of the vector in a host cell. They may be included as part of commercially available prokaryotic vectors and may also be chemically synthesized based on a known sequence and ligated into the vector. Various viral origins (e.g., SV40, polyoma, adenovirus, vesicular stomatitis virus (VSV), or papillomaviruses such as HPV or BPV) are useful for cloning vectors in mammalian cells.

Transcriptional and translational control sequences for mammalian host cell expression vectors can be excised from viral genomes. Commonly used promoter and enhancer sequences are derived from polyoma virus, adenovirus 2, simian virus 40 (SV40), and human cytomegalovirus (CMV). For example, the human CMV promoter/enhancer of immediate early gene 1 may be used. See e.g. Patterson et al., 1994, Applied Microbiol. Biotechnol. 40:691-98. DNA sequences derived from the SV40 viral genome, for example, SV40 origin, early and late promoter, enhancer, splice, and polyadenylation sites can be used to provide other genetic elements for expression of a structural gene sequence in a mammalian host cell. Viral early and late promoters are particularly useful because both are easily obtained from a viral genome as a fragment, which can also contain a viral origin of replication (Fiers et al., 1978, Nature 273:113; Kaufman, 1990, Meth. in Enzymol. 185:487-511). Smaller or larger SV40 fragments can also be used, provided the approximately 250 bp sequence extending from the Hind III site toward the BgII site located in the SV40 viral origin of replication site is included.

An enhancer sequence may be inserted into the vector to increase transcription by higher eukaryotes. Enhancers are cis-acting elements of DNA, usually about 10-300 bp in length, that act on the promoter to increase transcription. Enhancers are relatively orientation and position independent, having been found at positions both 5′ and 3′ to the transcription unit. Several enhancer sequences available from mammalian genes are known (e.g., globin, elastase, albumin, alpha-feto-protein and insulin). Typically, however, an enhancer from a virus is used. The SV40 enhancer, the cytomegalovirus early promoter enhancer, the polyoma enhancer, and adenovirus enhancers known in the art are exemplary enhancing elements for the activation of eukaryotic promoters. While an enhancer may be positioned in the vector either 5′ or 3′ to a coding sequence, it is typically located at a site 5′ from the promoter.

A transcription termination sequence is typically located 3′ to the end of a polypeptide coding region and serves to terminate transcription. Usually, a transcription termination sequence in prokaryotic cells is a G-C rich fragment followed by a poly-T sequence. While the sequence is easily cloned from a library or even purchased commercially as part of a vector, it can also be readily synthesized using methods for nucleic acid synthesis known to those of skill in the art.

In some cases, such as where glycosylation is desired in a eukaryotic host cell expression system, one may manipulate the various pre- or pro-sequences to improve glycosylation or yield. For example, one may alter the peptidase cleavage site of a particular signal peptide, or add prosequences, which also may affect glycosylation. The final protein product may have, in the −1 position (relative to the first amino acid of the mature protein), one or more additional amino acids incident to expression, which may not have been totally removed. For example, the final protein product may have one or two amino acid residues found in the peptidase cleavage site, attached to the amino-terminus. Alternatively, use of some enzyme cleavage sites may result in a slightly truncated form of the desired polypeptide if the enzyme cuts at such area within the mature polypeptide.

A sequence encoding an appropriate native or heterologous signal sequence (leader sequence or signal peptide) can be incorporated into an expression vector, to promote extracellular secretion of the protein of interest. The choice of signal peptide or leader depends on the type of host cells in which the protein of interest to be produced, and a heterologous signal sequence can replace the native signal sequence. Examples of signal peptides that are functional in mammalian host cells include the following: the signal sequence for interleukin-7 described in U.S. Pat. No. 4,965,195; the signal sequence for interleukin-2 receptor described in Cosman et al., 1984, Nature 312:768; the interleukin-4 receptor signal peptide described in EP Patent No. 0367 566; the type I interleukin-1 receptor signal peptide described in U.S. Pat. No. 4,968,607; the type II interleukin-1 receptor signal peptide described in EP Patent No. 0 460 846.

Additional control sequences shown to improve expression of heterologous genes from mammalian expression vectors include such elements as the expression augmenting sequence element (EASE) derived from CHO cells (Morris et al., in Animal Cell Technology, pp. 529-534 (1997); U.S. Pat. Nos. 6,312,951 B1, 6,027,915, and 6,309,841 B1) and the tripartite leader (TPL) and VA gene RNAs from Adenovirus 2 (Gingeras et al., 1982, J. Biol. Chem. 257:13475-13491). The internal ribosome entry site (IRES) sequences of viral origin allows bicistronic mRNAs to be translated efficiently (Oh and Sarnow, 1993, Current Opinion in Genetics and Development 3:295-300; Ramesh et al., 1996, Nucleic Acids Research 24:2697-2700).

### Proteins of Interest

Polypeptides and proteins of interest can be of scientific or commercial interest, including protein-based therapeutics. Proteins of interest include, among other things, secreted proteins, non-secreted proteins, intracellular proteins or membrane-bound proteins. Polypeptides and proteins of interest can be produced by recombinant animal cell lines using cell culture methods and may be referred to as “recombinant proteins”. The expressed protein(s) may be produced intracellularly or secreted into the culture medium from which it can be recovered and/or collected. The term “isolated protein” or “isolated recombinant protein” refers to a polypeptide or protein of interest, that is purified away from proteins or polypeptides or other contaminants that would interfere with its therapeutic, diagnostic, prophylactic, research or other use. Proteins of interest include proteins that exert a therapeutic effect by binding a target, particularly a target among those listed below, including targets derived therefrom, targets related thereto, and modifications thereof.

Proteins of interest include “antigen-binding proteins”. Antigen-binding protein refers to proteins or polypeptides that comprise an antigen-binding region or antigen-binding portion that has affinity for another molecule to which it binds (antigen). Antigen-binding proteins encompass antibodies, peptibodies, antibody fragments, antibody derivatives, antibody analogs, Fc fusion proteins (including single-chain variable fragments (scFvs)), double-chain (divalent) scFvs, and IgGscFv (see, e.g., Orcutt et al., 2010, Protein Eng Des2 Sel 23:221-228), heteroIgG (see, e.g., Liu et al., 2015, J Biol Chem 290:7535-7562), muteins, and XmAb® (Xencor, Inc., Monrovia, CA). Examples of antigen binding proteins include a human antibody, a humanized antibody; a chimeric antibody; a recombinant antibody; a single chain antibody; a diabody; a triabody; a tetrabody; a Fab fragment; a F(ab′)₂ fragment; an IgD antibody; an IgE antibody; an IgM antibody; an IgG1 antibody; an IgG2 antibody; an IgG3 antibody; or an IgG4 antibody, and fragments thereof. Also included are bispecific T cell engagers (BITE®), bispecific T cell engagers having extensions, such as half-life extensions, for example HLE BiTEs, HeteroIg BITE and others.

As used herein, the term “antigen binding protein” is used in its broadest sense and means a protein comprising a portion that binds to an antigen or target and, optionally, a scaffold or framework portion that allows the antigen binding portion to adopt a conformation that promotes binding of the antigen binding protein to the antigen. The antigen binding protein can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen binding protein as well as wholly synthetic scaffolds comprising, for example, a biocompatible polymer. See, e.g., Korndorfer et al., 2003, Proteins: Structure, Function, and Bioinformatics, 53 (1): 121-129; Roque et al., 2004, Biotechnol. Prog. 20:639-654. In addition, peptide antibody mimetics (“PAMs”) can be used, as well as scaffolds based on antibody mimetics utilizing fibronectin components as a scaffold.

An antigen binding protein can have, for example, the structure of a naturally occurring immunoglobulin. An “immunoglobulin” is a tetrameric molecule. In a naturally occurring immunoglobulin, each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgG, IgA, and IgE, respectively.

Naturally occurring immunoglobulin chains exhibit the same general structure of relatively conserved framework regions (FR) joined by three hypervariable regions, also called complementarity determining regions or CDRs. From N-terminus to C-terminus, both light and heavy chains comprise the domains FR1, CDR1, FR2, CDR2, FR3, CDR3 and FR4. The assignment of amino acids to each domain can be done in accordance with the definitions of Kabat et al. in Sequences of Proteins of Immunological Interest, 5^{th} Ed., US Dept. of Health and Human Services, PHS, NIH, NIH Publication no. 91-3242, (1991). As desired, the CDRs can also be redefined according to an alternative nomenclature scheme, such as that of Chothia (see Chothia and Lesk, 1987, J. Mol. Biol. 196:901-917; Chothia et al., 1989, Nature 342:878-883 or Honegger and Pluckthun, 2001, J. Mol. Biol. 309:657-670).

In the context of the instant disclosure, an antigen binding protein is said to “specifically bind” or “selectively bind” its target antigen when the dissociation constant (K_{D}) is ≤10⁻⁸ M. The antibody specifically binds antigen with “high affinity” when the K_{D} is ≤5×10⁻⁹ M, and with “very high affinity” when the K_{D} is ≤5×10⁻¹⁰ M.

The term “antibody” includes reference to both glycosylated and non-glycosylated immunoglobulins of any isotype or subclass or to an antigen-binding region thereof that competes with the intact antibody for specific binding, unless otherwise specified. Additionally, the term “antibody” refers to an intact immunoglobulin that competes with the intact antibody for specific binding, unless otherwise specified.

Antigen binding portions can be produced by recombinant DNA techniques or by enzymatic or chemical cleavage of intact antibodies and can form an element of a protein of interest. Unless otherwise specified, antibodies include human, humanized, chimeric, multi-specific, monoclonal, polyclonal, heteroIgG, bispecific, and oligomers or antigen binding fragments thereof. Antibodies include the lgG1-, lgG2-lgG3- or lgG4-type. Also included are proteins having an antigen binding fragment or region such as Fab, Fab′, F(ab′)2, Fv, diabodies, Fd, dAb, maxibodies, single chain antibody molecules, single domain V_{H}H, complementarity determining region (CDR) fragments, scFv, diabodies, triabodies, tetrabodies and polypeptides that contain at least a portion of an immunoglobulin that is sufficient to confer specific antigen binding to a target polypeptide.

An antigen binding protein can have one or more binding sites. If there is more than one binding site, the binding sites can be identical to one another or can be different. For example, a naturally occurring human immunoglobulin typically has two identical binding sites, while a “bispecific” or “bifunctional” antibody has two different binding sites.

A Fab fragment is a monovalent fragment having the V_{L}, V_{H}, C_{L} and C_{H}1 domains; a F(ab′)₂ fragment is a bivalent fragment having two Fab fragments linked by a disulfide bridge at the hinge region; a Fd fragment has the V_{H} and C_{H}1 domains; an Fv fragment has the V_{L} and V_{H} domains of a single arm of an antibody; and a dAb fragment has a V_{H} domain, a V_{L} domain, or an antigen-binding fragment of a V_{H} or V_{L} domain (U.S. Pat. Nos. 6,846,634, 6,696,245, U.S. Patent Application Publication Nos. 2005/0202512, 2004/0202995, 2004/0038291, 2004/0009507, 2003/0039958, Ward et al., 1989, Nature 341:544-546).

A single-chain antibody (scFv) is an antibody in which a V_{L} and a V_{H} region are joined via a linker (e.g., a synthetic sequence of amino acid residues) to form a continuous protein chain wherein the linker is long enough to allow the protein chain to fold back on itself and form a monovalent antigen binding site (see, e.g., Bird et al., 1988, Science 242:423-26 and Huston et al., 1988, Proc. Natl. Acad. Sci. USA 85:5879-83), U.S. Pat. Nos. 7,741,465, and 6,319,494 as well as Eshhar et al., 1997, Cancer Immunol Immunotherapy 45:131-136. An scFv retains the parent antibody's ability to specifically interact with target antigen.

Diabodies are bivalent antibodies comprising two polypeptide chains, wherein each polypeptide chain comprises V_{H} and V_{L} domains joined by a linker that is too short to allow for pairing between two domains on the same chain, thus allowing each domain to pair with a complementary domain on another polypeptide chain (see, e.g., Holliger et al., 1993, Proc. Natl. Acad. Sci. USA 90:6444-48; and Poljak et al., 1994, Structure 2:1121-23). If the two polypeptide chains of a diabody are identical, then a diabody resulting from their pairing will have two identical antigen binding sites. Polypeptide chains having different sequences can be used to make a diabody with two different antigen binding sites. Similarly, tribodies and tetrabodies are antibodies comprising three and four polypeptide chains, respectively, and forming three and four antigen binding sites, respectively, which can be the same or different.

For purposes of clarity, and as described herein, it is noted that an antigen binding protein can, but need not, be of human origin (e.g., a human antibody), and in some cases will comprise a non-human protein, for example a rat or murine protein, and in other cases an antigen binding protein can comprise a hybrid of human and non-human proteins (e.g., a humanized antibody).

A protein of interest can comprise a human antibody. The term “human antibody” includes all antibodies that have one or more variable and constant regions derived from human immunoglobulin sequences. In one embodiment, all of the variable and constant domains are derived from human immunoglobulin sequences (a fully human antibody). Such antibodies can be prepared in a variety of ways, including through the immunization with an antigen of interest of a mouse that is genetically modified to express antibodies derived from human heavy and/or light chain-encoding genes, such as a mouse derived from a Xenomouse®, UltiMab™, or Velocimmune® system, or a rat derived from UniRat®. Phage-based approaches can also be employed.

Alternatively, a protein of interest can comprise a humanized antibody. A “humanized antibody” has a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In one embodiment, certain amino acids in the framework and constant domains of the heavy and/or light chains of the non-human species antibody are mutated to produce the humanized antibody. In another embodiment, the constant domain(s) from a human antibody are fused to the variable domain(s) of a non-human species. Examples of how to make humanized antibodies can be found in U.S. Pat. Nos. 6,054,297, 5,886,152 and 5,877,293.

Also included are modified proteins, such as proteins modified chemically by a non-covalent bond, covalent bond, or both a covalent and non-covalent bond. Also included are proteins further comprising one or more post-translational modifications, which may be made by cellular modification systems or modifications introduced ex vivo by enzymatic and/or chemical methods or introduced in other ways.

In some embodiments, proteins of interest may include proteins that bind specifically to one or more CD proteins, HER receptor family proteins, cell adhesion molecules, growth factors, nerve growth factors, fibroblast growth factors, transforming growth factors (TGF), insulin-like growth factors, osteoinductive factors, insulin and insulin-related proteins, coagulation and coagulation-related proteins, colony stimulating factors (CSFs), other blood and serum proteins blood group antigens; receptors, receptor-associated proteins, growth hormones, growth hormone receptors, T-cell receptors; neurotrophic factors, neurotrophins, relaxins, interferons, interleukins, viral antigens, lipoproteins, integrins, rheumatoid factors, immunotoxins, surface membrane proteins, transport proteins, homing receptors, addressins, regulatory proteins, and immunoadhesins.

In some embodiments proteins of interest bind to one of more of the following, alone or in any combination: CD proteins including but not limited to CD3, CD4, CD5, CD7, CD8, CD19, CD20, CD22, CD25, CD30, CD33, CD34, CD38, CD40, CD70, CD123, CD133, CD138, CD171, and CD174, HER receptor family proteins, including, for instance, HER2, HER3, HER4, and the EGF receptor, EGFRvIII, cell adhesion molecules, for example, LFA-1, Mol, p150,95, VLA-4, ICAM-1, VCAM, and alpha v/beta 3 integrin, growth factors, including but not limited to, for example, vascular endothelial growth factor (“VEGF”); VEGFR2, growth hormone, thyroid stimulating hormone, follicle stimulating hormone, luteinizing hormone, growth hormone releasing factor, parathyroid hormone, mullerian-inhibiting substance, human macrophage inflammatory protein (MIP-1-alpha), erythropoietin (EPO), nerve growth factor, such as NGF-beta, platelet-derived growth factor (PDGF), fibroblast growth factors, including, for instance, aFGF and bFGF, epidermal growth factor (EGF), Cripto, transforming growth factors (TGF), including, among others, TGF-α and TGF-β, including TGF-β1, TGF-β2, TGF-β3, TGF-β4, or TGF-β5, insulin-like growth factors-I and -II (IGF-I and IGF-II), des(1-3)-IGF-I (brain IGF-I), and osteoinductive factors, insulins and insulin-related proteins, including but not limited to insulin, insulin A-chain, insulin B-chain, proinsulin, and insulin-like growth factor binding proteins; (coagulation and coagulation-related proteins, such as, among others, factor VIII, tissue factor, von Willebrand factor, protein C, alpha-1-antitrypsin, plasminogen activators, such as urokinase and tissue plasminogen activator (“t-PA”), bombazine, thrombin, thrombopoietin, and thrombopoietin receptor, colony stimulating factors (CSFs), including the following, among others, M-CSF. GM-CSF, and G-CSF, other blood and serum proteins, including but not limited to albumin, IgE, and blood group antigens, receptors and receptor-associated proteins, including, for example, flk2/flt3 receptor, obesity (OB) receptor, growth hormone receptors, and T-cell receptors; neurotrophic factors, including but not limited to, bone-derived neurotrophic factor (BDNF) and neurotrophin-3, -4, -5, or -6 (NT-3, NT-4, NT-5, or NT-6); relaxin A-chain, relaxin B-chain, and prorelaxin, interferons, including for example, interferon-alpha, -beta, and -gamma, interleukins (ILs), e.g., IL-1 to IL-10, IL-12, IL-15, IL-17, IL-23, IL-12/IL-23, IL-2Ra, IL1-R1, IL-6 receptor, IL-4 receptor and/or IL-13 to the receptor, IL-13RA2, or IL-17 receptor, IL-1RAP; viral antigens, including but not limited to, an AIDS envelope viral antigen, lipoproteins, calcitonin, glucagon, atrial natriuretic factor, lung surfactant, tumor necrosis factor-alpha and -beta, enkephalinase, BCMA, IgKappa, ROR-1, ERBB2, mesothelin, RANTES (regulated on activation normally T-cell expressed and secreted), mouse gonadotropin-associated peptide. DNase, FR-alpha, inhibin, and activin, integrin, protein A or D, rheumatoid factors, immunotoxins, bone morphogenetic protein (BMP), superoxide dismutase, surface membrane proteins, decay accelerating factor (DAF), AIDS envelope, transport proteins, homing receptors, MIC (MIC-a, MIC-B), ULBP 1-6, EPCAM, addressins, regulatory proteins, immunoadhesins, antigen-binding proteins, somatropin, CTGF, CTLA4, cotaxin-1, MUC1, CEA, c-MET, Claudin-18, GPC-3, EPHA2, FPA, LMP1, MG7, NY-ESO-1, PSCA, ganglioside GD2, ganglioside GM2, BAFF, OPGL (RANKL), myostatin, Dickkopf-1 (DKK-1), Ang2, NGF, IGF-1 receptor, hepatocyte growth factor (HGF), TRAIL-R2, c-Kit, B7RP-1, PSMA, NKG2D-1, programmed cell death protein 1 and ligand, PD1 and PDL1, mannose receptor/hCGβ, hepatitis-C virus, mesothelin dsFv[PE38] conjugate, Legionella pneumophila (lly), IFN gamma, interferon gamma induced protein 10 (IP10), IFNAR, TALL-1, thymic stromal lymphopoietin (TSLP), proprotein convertase subtilisin/Kexin Type 9 (PCSK9), stem cell factors, Flt-3, calcitonin gene-related peptide (CGRP), OX40 L, α4β7, platelet specific (platelet glycoprotein IIb/IIIb (PAC-1), transforming growth factor beta (TFGβ), Zona pellucida sperm-binding protein 3 (ZP-3), TWEAK, platelet derived growth factor receptor alpha (PDGFRα), sclerostin, and biologically active fragments or variants of any of the foregoing.

In another embodiment, proteins of interest include abciximab, adalimumab, adecatumumab, aflibercept, alemtuzumab, alirocumab, anakinra, atacicept, basiliximab, belimumab, bevacizumab, biosozumab, blinatumomab, brentuximab vedotin, brodalumab, cantuzumab mertansine, canakinumab, cetuximab, certolizumab pegol, conatumumab, daclizumab, denosumab, eculizumab, edrecolomab, efalizumab, epratuzumab, etanercept, evolocumab, galiximab, ganitumab, gemtuzumab, golimumab, ibritumomab tiuxetan, infliximab, ipilimumab, ixekizumab lerdelimumab, lumiliximab, mapatumumab, motesanib diphosphate, muromonab-CD3, natalizumab, nesiritide, nimotuzumab, nivolumab, ocrelizumab, ofatumumab, omalizumab, oprelvekin, palivizumab, panitumumab, pembrolizumab, pertuzumab, pexelizumab, ranibizumab, rilotumumab, rituximab, romiplostim, romosozumab, sargamostim, tocilizumab, tositumomab, trastuzumab, ustekinumab, vedolizumab, visilizumab, volociximab, zanolimumab, zalutumumab, and biosimilars of any of the foregoing.

Proteins of interest according to the invention encompass all of the foregoing and further include antibodies comprising 1, 2, 3, 4, 5, or 6 of the complementarity determining regions (CDRs) of any of the aforementioned antibodies. One or more CDRs can be incorporated into a molecule either covalently or noncovalently to make it an antigen binding protein. An antigen binding protein can incorporate the CDR(s) as part of a larger polypeptide chain, can covalently link the CDR(s) to another polypeptide chain, or can incorporate the CDR(s) noncovalently. The CDRs permit the antigen binding protein to specifically bind to a particular antigen of interest. Also included are variants that comprise a region that is 70% or more, especially 80% or more, more especially 90% or more, yet more especially 95% or more, particularly 97% or more, more particularly 98% or more, yet more particularly 99% or more identical in amino acid sequence to a reference amino acid sequence of a protein of interest. Identity in this regard can be determined using a variety of well-known and readily available amino acid sequence analysis software. Preferred software includes those that implement the Smith-Waterman algorithms, considered a satisfactory solution to the problem of searching and aligning sequences. Other algorithms also may be employed, particularly where speed is an important consideration. Commonly employed programs for alignment and homology matching of DNAs, RNAs, and polypeptides that can be used in this regard include FASTA, TFASTA, BLASTN, BLASTP, BLASTX, TBLASTN, PROSRCH, BLAZE, and MPSRCH, the latter being an implementation of the Smith-Waterman algorithm for execution on massively parallel processors made by MasPar.

Preferably, the antigen binding molecule is an antibody fragment thereof, and more preferably one or more single chain antibody fragment (“scFv”). scFvs are preferred for use in chimeric antigen receptors because they can be engineered to be expressed as part of a single chain. See Krause et al., 1988, J. Exp. Med., 188 (4): 619-626; Finney et al., 1998, J Immunol 161:2791-2797.

An “Fc” region, as the term is used herein, comprises two heavy chain fragments comprising the C_{H}2 and C_{H}3 domains of an antibody. The two heavy chain fragments are held together by two or more disulfide bonds and by hydrophobic interactions of the C_{H}3 domains. Proteins of interest comprising an Fc region, including antigen binding proteins and Fc fusion proteins, form another aspect of the instant disclosure.

A “hemibody” is an immunologically functional immunoglobulin construct comprising a complete heavy chain, a complete light chain and a second heavy chain Fc region paired with the Fc region of the complete heavy chain. A linker can, but need not, be employed to join the heavy chain Fc region and the second heavy chain Fc region. In particular embodiments, a hemibody is a monovalent form of an antigen binding protein disclosed herein. In other embodiments, pairs of charged residues can be employed to associate one Fc region with the second Fc region. A hemibody can be a protein of interest in the context of the instant disclosure.

### Generation of Mammalian Host Cells Expressing a Protein of Interest

Expression of a protein of interest in a cell can be achieved by well-known methods, either transiently or by stable expression (Davis et al., Basic Methods in Molecular Biology, 2^{nd} ed., Appleton & Lange, Norwalk, Conn., 1994; Sambrook et al., Molecular Cloning: A Laboratory Manual, 3rd ed., Cold Spring Harbor Laboratory Press, Cold Spring Harbor, N. Y., 2001).

Methods for stable integration are well known in the art. Briefly, stable integration is commonly achieved by transiently introducing a heterologous polynucleotide or a vector containing the heterologous polynucleotide into the host cell, which facilitates the stable integration of said heterologous polynucleotide into the cell genome. Typically, the heterologous polynucleotide is flanked by homology arms, i.e., sequences homologous to the region upstream and downstream to the integration site. Before their introduction into the mammalian host cell, circular vectors may be linearized to facilitate integration into the cell genome. Methods for the introduction of vectors into cells are well known in the art and include transfection with biological methods, such as viral delivery, with chemical methods, such as using cationic polymers, calcium phosphate, cationic lipids or cationic amino acids; with physical methods, such as electroporation or microinjection; or with mixed approaches, such as protoplast fusion.

A specific method of stable integration uses recombinase mediated cassette exchange (RMCE; Bode and Baer, 2001, Curr Opin Biotechnol. 12:473-80, and Bode et al., 2000, Biol. Chem. 381:801-813) for site-specific integration in the genome (also termed “targeted integration”). Site-specific recombinases such as Flp and Cre mediate recombination between two copies of their target sequence termed FRT and loxP, respectively. The use of two incompatible target sequences, for example FRT in combination with F3 (Schlake and Bode, 1994, Biochemistry, 33:12746-51) as well as inverted recognition target sites (Feng et al., 1999. J. Mol. Biol. 292:779-85) allows the insertion of DNA segments into a predefined chromosomal locus carrying target sequences in a similar configuration. See also EP Patent No. EP1781796B1 and EP Patent Application Publication No. EP2789691A1.

Insertion of RMCE into a specific site in the genome can be mediated by nucleases (e.g., zinc finger protein (ZFP), transcription activator-like effector nuclease (TALEN), clustered regularly interspaced short palindromic repeats (CRISPR)/CRISPR-associated protein 9 (Cas9)) that can be engineered to create single- and double-stranded breaks (SSBs/DSBs) in the genome. There are two major and distinct pathways to repair DSBs—homologous recombination and non-homologous end-joining (NHEJ). Homologous recombination requires the presence of a homologous sequence as a template (e.g., “donor” containing RMCE) to guide the cellular repair process and the results of the repair are error-free and predictable. In the absence of a template (or “donor”) sequence for homologous recombination, the cell typically attempts to repair the DSB via the unpredictable and error-prone process of non-homologous end-joining (NHEJ).

A vector may be any molecule or entity (e.g., nucleic acid, plasmid, bacteriophage, transposon, cosmid, chromosome, virus, virus capsid, virion, naked DNA, complexed DNA and the like) suitable for use to transfer and/or transport protein encoding information into a host cell and/or to a specific location and/or compartment within a host cell. Vectors can include viral and non-viral vectors, non-episomal mammalian vectors. Vectors are often referred to as expression vectors, for example, recombinant expression vectors and cloning vectors. The vector may be introduced into a host cell to allow replication of the vector itself and thereby amplify the copies of the polynucleotide contained therein. The cloning vectors may contain sequence components generally include, without limitation, an origin of replication, promoter sequences, transcription initiation sequences, enhancer sequences, and selectable markers. These elements may be selected as appropriate by a person of ordinary skill in the art.

Following construction, one or more vectors may be inserted into a suitable cell for amplification and/or polypeptide expression. The transformation of an expression vector into a selected cell may be accomplished by well-known methods including transfection, infection, calcium phosphate co-precipitation, electroporation, nucleofection, microinjection, DEAE-dextran mediated transfection, cationic lipids mediated delivery, liposome mediated transfection, microprojectile bombardment, receptor-mediated gene delivery, delivery mediated by polylysine, histone, chitosan, and peptides. The method selected will in part be a function of the type of host cell to be used. These methods and other suitable methods are well known to the skilled artisan and are set forth in manuals and other technical publications, for example, in Sambrook et al., Molecular Cloning: A Laboratory Manual, 3rd ed., Cold Spring Harbor Laboratory Press, Cold Spring Harbor, N.Y. (2001).

The term “transformation” refers to a change in a cell's genetic characteristics, and a cell has been transformed when it has been modified to contain new DNA or RNA. For example, a cell is transformed where it is genetically modified from its native state by introducing new genetic material via transfection, transduction, or other techniques. Following transfection or transduction, the transforming DNA can recombine with that of the cell by physically integrating into a chromosome of the cell or can be maintained transiently as an episomal element without being replicated or can replicate independently as a plasmid. A cell is considered to have been “stably transformed” when the transforming DNA is replicated with the division of the cell.

The term “transfection” refers to the uptake of foreign or exogenous DNA by a cell. A number of transfection techniques are well known in the art and are disclosed herein. See, e.g., Graham et al., 1973, Virology 52:456; Sambrook et al., 2001, Molecular Cloning: A Laboratory Manual, supra; Davis et al., 1986, Basic Methods in Molecular Biology, Elsevier; Chu et al., 1981, Gene 13:197.

The term “transduction” refers to the process whereby foreign DNA is introduced into a cell via viral vector. See Jones et al., (1998). Genetics: principles and analysis. Boston: Jones & Bartlett Publ.

### Cell Lines

In the methods disclosed herein, any mammalian cell line can be used. A wide variety of mammalian cell lines suitable for growth in culture are available from the American Type Culture Collection (Manassas, Va.) and commercial vendors. Examples of cell lines commonly used in the industry include monkey kidney CVI line transformed by SV40 (COS-7, ATCC CRL 1651); human embryonic kidney line (293 or 293 cells subcloned for growth in suspension culture, (Graham et al, 1977, J. Gen Virol. 36:59); baby hamster kidney cells (BHK, ATCC CCL 10); mouse Sertoli cells (TM4, Mather, 1980, Biol. Reprod. 23:243-251); monkey kidney cells (CVI ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human hepatoma cells (Hep G2, HB 8065); mouse mammary tumor (MMT 060562, ATCC CCL51); TRI cells (Mather et al., 1982, Annals N. Y Acad. Sci. 383:44-68); MRC 5 cells or FS4 cells; mammalian myeloma cells, and a number of other cell lines and Chinese hamster ovary (CHO) cells.

Large-scale production of proteins for commercial applications is typically carried out in suspension culture. Therefore, mammalian host cells used to generate the recombinant mammalian cells described herein can, but need not be, adapted to growth in suspension culture. A variety of host cells adapted to growth in suspension culture are known, including mouse myeloma NS0 cells and CHO cells from CHO-S, DG44, and DXB11 cell lines. Other suitable cell lines include mouse myeloma SP2/0 cells, baby hamster kidney BHK-21 cells, human PER.C6® cells, human embryonic kidney HEK-293 cells, and cell lines derived or engineered from any of the cell lines disclosed herein.

CHO cells are widely used to produce complex recombinant proteins, including CHOK1 cells (ATCC CCL61). The dihydrofolate reductase (DHFR)-deficient mutant cell lines (Urlaub et al., 1980, Proc Natl Acad Sci USA 77:4216-4220), DXB11 and DG-44, are desirable CHO host cell lines because the efficient DHFR selectable and amplifiable gene expression system allows high level recombinant protein expression in these cells (Kaufman R. J., 1990, Meth Enzymol 185:537-566). Also included are the glutamine synthase (GS)-knockout CHOK1SV cell lines, making use of glutamine synthetase (GS)-based methionine sulfoximine (MSX) selection. Other suitable CHO host cells could include, but are not limited to the following (ECACC accession numbers in brackets): CHO (85050302), CHO (PROTEIN FREE) (00102307), CHO-K1 (85051005), CHO-K1/SF (93061607), CHO/DHFR-(94060607), CHO/DHFR-AC-free (05011002), RR-CHOKI (92052129).

### Cell Culture Process

Host cells transfected with the vector systems described herein to adherent culture or suspension cultures grown in stirred tank reactors (including traditional batch and fed-batch cell cultures, which may but need not comprise a spin filter), perfusion systems (including alternating tangential flow (“ATF”) cultures, acoustic perfusion systems, depth filter perfusion systems, and other systems), hollow fiber bioreactors (HFB, which in some cases may be employed in perfusion processes) as well as various other cell culture methods (see, e.g., Tao et al., 2003, Biotechnol. Bioeng. 82:751-65; Kuystermans & Al-Rubeai, (2011) “Bioreactor Systems for Producing Antibody from Mammalian Cells” in Antibody Expression and Production, Cell Engineering 7:25-52. Al-Rubeai (ed) Springer; Catapano et al., (2009) “Bioreactor Design and Scale-Up” in Cell and Tissue Reaction Engineering: Principles and Practice, Eibl et al. (eds) Springer-Verlag, incorporated herein by reference in their entireties).

During recombinant protein production it is desirable to have a controlled system where cells are grown to a desired density and then the physiological state of the cells is switched to a growth-arrested, high productivity state where the cells use energy and substrates to produce the recombinant protein of interest instead of making more cells. Various methods for accomplishing this goal exist, and include temperature shifts and amino acid starvation, as well as use of a cell-cycle inhibitor or other molecule that can arrest cell growth without causing cell death.

The production of a recombinant protein begins with establishing a mammalian cell production culture of cells that express the protein, in a culture plate, flask, tube, bioreactor or other suitable vessel. Smaller production bioreactors are typically used, in one embodiment the bioreactors are 500 L to 2000 L. In another embodiment, 1000 L-2000 L bioreactors are used. The seed cell density used to inoculate the bioreactor can have a positive impact on the level of recombinant protein produced. In one embodiment the bioreactor is inoculated with at least 0.5×10⁶ up to and beyond 3.0×10⁶ viable cells/mL in a serum-free culture medium. In another embodiment the inoculation is 1.0×10⁶ viable cells/mL.

The mammalian cells then undergo an exponential growth phase. The cell culture can be maintained without supplemental feeding until a desired cell density is achieved. In one embodiment the cell culture is maintained for up to three days with or without supplemental feeding. In another embodiment the culture can be inoculated at a desired cell density to begin the production phase without a brief growth phase. In any of the embodiments herein the switch from the growth phase to production phase can also be initiated by any of the aforementioned methods.

Three methods are typically used in commercial processes for the production of recombinant proteins by mammalian cell culture: batch culture, fed-batch culture, and perfusion culture. Batch culture is a discontinuous method where cells are grown in a fixed volume of culture media for a short period of time followed by a full harvest. Cultures grown using the batch method experience an increase in cell density until a maximum cell density is reached, followed by a decline in viable cell density as the media components are consumed and levels of metabolic by-products (such as lactate and ammonia) accumulate. Harvest typically occurs at the point when the maximum cell density is achieved (e.g., 5×10⁶ cells/mL or greater, depending on media formulation, cell line, etc.). The batch process is the simplest culture method, however viable cell density is limited by the nutrient availability and once the cells are at maximum density, the culture declines and production decreases. There is no ability to extend a production phase because the accumulation of waste products and nutrient depletion rapidly lead to culture decline, (typically around 3 to 7 days).

Fed-batch culture improves on the batch process by providing bolus or continuous media feeds to replenish those media components that have been consumed. Since fed-batch cultures receive additional nutrients throughout the run, they have the potential to achieve higher cell densities (>10 to 30×10⁶ cells/ml, depending on media formulation, cell line, etc.) and increased product titers, when compared to the batch method. Unlike the batch process, a biphasic culture can be created and sustained by manipulating feeding strategies and media formulations to distinguish the period of cell proliferation to achieve a desired cell density (the growth phase) from the period of suspended or slow cell growth (the production phase). As such, fed batch cultures have the potential to achieve higher product titers compared to batch cultures. Typically, a batch method is used during the growth phase and a fed-batch method used during the production phase, but a fed-batch feeding strategy can be used throughout the entire process. However, unlike the batch process, bioreactor volume is a limiting factor which limits the amount of feed. Also, as with the batch method, metabolic by-product accumulation will lead to culture decline, which limits the duration of the production phase, about 10 to 21 days. Fed-batch cultures are discontinuous, and harvest typically occurs when metabolic by-product levels or culture viability reach predetermined levels. When compared to a batch culture, in which no feeding occurs, a fed batch culture can produce greater amounts of recombinant protein. See e.g. U.S. Pat. No. 5,672,502.

Perfusion methods offer potential improvement over the batch and fed-batch methods by adding fresh media and simultaneously removing spent media. Typical large scale commercial cell culture strategies strive to reach high cell densities, 60-90 (+)×10⁶ cells/mL where almost a third to over one-half of the reactor volume is biomass. With perfusion culture, extreme cell densities of >1×10⁸ cells/mL have been achieved and even higher densities are predicted. Typical perfusion cultures begin with a batch culture start-up lasting for a day or two followed by continuous, step-wise and/or intermittent addition of fresh feed media to the culture and simultaneous removal of spent media with the retention of cells and additional high molecular weight compounds such as proteins (based on the filter molecular weight cutoff) throughout the growth and production phases of the culture. Various methods, such as sedimentation, centrifugation, or filtration, can be used to remove spent media, while maintaining cell density. Perfusion flow rates of a fraction of a working volume per day up to many multiple working volumes per day have been reported.

An advantage of the perfusion process is that the production culture can be maintained for longer periods than batch or fed-batch culture methods. However, increased media preparation, use, storage and disposal are necessary to support a long-term perfusion culture, particularly those with high cell densities, which also need even more nutrients, and all of this drives the production costs even higher, compared to batch and fed batch methods. In addition, higher cell densities can cause problems during production, such as maintaining dissolved oxygen levels and problems with increased gassing including supplying more oxygen and removing more carbon dioxide, which would result in more foaming and the need for alterations to antifoam strategies; as well as during harvest and downstream processing where the efforts required to remove the excessive cell material can result in loss of product, negating the benefit of increased titer due to increased cell mass.

Also provided is a large-scale cell culture strategy that combines fed batch feeding during the growth phase followed by continuous perfusion during the production phase. The method targets a production phase where the cell culture is maintained at a packed cell volume of less than or equal to 35%.

In one embodiment, a fed-batch culture with bolus feeds is used to maintain a cell culture during the growth phase. Perfusion feeding can then be used during a production phase. In one embodiment, perfusion begins when the cells have reached a production phase. In another embodiment, perfusion begins on or about day 3 to on or about day 9 of the cell culture. In another embodiment perfusion begins on or about day 5 to on or about day 7 of the cell culture.

Using bolus feeding during the growth phase allows the cells to transition into the production phase, resulting in less dependence on a temperature shift as a means of initiating and controlling the production phase, however a temperature shift of about 36° C. to about 31° C. can take place between the growth phase and production phase. In one embodiment the shift is from 36° C. to 32° C.

As described herein, the bioreactor can be inoculated with at least 0.5×10⁶ up to and beyond 3.0×10⁶ viable cells/mL in a serum-free culture medium, for example 1.0×10⁶ viable cells/mL.

Perfusion culture is one in which the cell culture receives fresh perfusion feed medium while simultaneously removing spent medium. Perfusion can be continuous, stepwise, intermittent, or a combination of any or all of any of these. Perfusion rates can be less than a working volume to many working volumes per day. The cells are retained in the culture and the spent medium that is removed is substantially free of cells or has significantly fewer cells than the culture. Recombinant proteins expressed by the cell culture can also be retained in the culture. Perfusion can be accomplished by a number of means including centrifugation, sedimentation, or filtration, See e.g. Voisard et al., 2003, Biotechnology and Bioengineering 82:751-65. An example of a filtration method is alternating tangential flow filtration. Alternating tangential flow is maintained by pumping medium through hollow-fiber filter modules. See e.g. U.S. Pat. No. 6,544,424; Furey, 2002, Gen. Eng. News. 22 (7): 62-63.

“Perfusion flow rate” is the amount of media that is passed through (added and removed) from a bioreactor, typically expressed as some portion or multiple of the working volume, in a given time. “Working volume” refers to the amount of bioreactor volume used for cell culture. In one embodiment the perfusion flow rate is one working volume or less per day. Perfusion feed medium can be formulated to maximize perfusion nutrient concentration to minimize perfusion rate.

Cell cultures can be supplemented with concentrated feed medium containing components, such as nutrients and amino acids, which are consumed during the course of the production phase of the cell culture.

Concentrated feed medium may be based on just about any cell culture media formulation. Such a concentrated feed medium can contain most of the components of the cell culture medium at, for example, about 5×, 6×, 7×, 8×, 9×, 10×, 12×, 14×, 16×, 20×, 30×, 50×, 100×, 200×, 400×, 600×, 800×, or even about 1000× of their normal amount. Concentrated feed media are often used in fed batch culture processes.

Samples from the cell culture can be monitored and evaluated using any of the analytical techniques known in the art. A variety of parameters including recombinant protein and medium quality and characteristics can be monitored for the duration of the culture. Samples can be taken and monitored intermittently at a desirable frequency, including continuous monitoring, real time or near real time.

Typically, the cell cultures that precede the final production culture (N−x to N−1) are used to generate the seed cells that will be used to inoculate the production bioreactor, the N−1 culture. The seed cell density can have a positive impact on the level of recombinant protein produced. Product levels tend to increase with increasing seed density. Improvement in titer is tied not only to higher seed density, but is likely to be influenced by the metabolic and cell cycle state of the cells that are placed into production.

Seed cells can be produced by any culture method. One such method is a perfusion culture using alternating tangential flow filtration. An N−1 bioreactor can be run using alternating tangential flow filtration to provide cells at high density to inoculate a production bioreactor. The N−1 stage may be used to grow cells to densities of >90×10⁶ cells/mL. The N−1 bioreactor can be used to generate bolus seed cultures or can be used as a rolling seed stock culture that could be maintained to seed multiple production bioreactors at high seed cell density. The duration of the growth stage of production can range from 7 to 14 days and can be designed so as to maintain cells in exponential growth prior to inoculation of the production bioreactor. Perfusion rates, medium formulation and timing are optimized to grow cells and deliver them to the production bioreactor in a state that is most conducive to optimizing their production. Seed cell densities of >15×10⁶ cells/mL can be achieved for seeding production bioreactors. Higher seed cell densities at inoculation can decrease or even eliminate the time needed to reach a desired production density.

In certain embodiments, the mammalian host cells can be used to generate high yield of a protein of interest. High yield, or high volumetric productivity, to the ability of cells to produce high levels of a protein of interest. The particular yield will depend on the protein of interest and can be at least 0.05 g/L, at least 0.1 g/L, at least 0.15 g/L, at least 0.2 g/L, at least 0.25 g/L, at least 0.3 g/L, at least 0.35 g/L, at least 0.4 g/L, at least 0.45 g/L, at least 0.5 g/L, at least 0.6 g/L, at least 0.7 g/L, at least 0.8 g/L, at least 0.9 g/L, at least 1 g/L, at least 1.5 g/L, at least 2 g/L, or more, in a 10-day culture grown in fed batch or perfusion conditions, using a feed medium suitable for the mammalian host cell and containing amino acids, vitamins, or trace elements. In specific embodiments, the host cells and methods of the present disclosure express a protein of interest and are capable of producing at least 0.5 g/L, at least 0.6 g/L, at least 0.7 g/L, at least 0.8 g/L, at least 0.9 g/L, at least 1 g/L, at least 1.5 g/L, at least 2 g/L, or more, preferably up to about 3 g/L, 4 g/L, 5 g/L or 10 g/L when grown under the culture conditions described above.

Yield can also be measured in terms of the specific productivity of a cell line, determined based on the amount of protein produced per cell per day (expressed as pg/cell/day). Mammalian host cells of the present disclosure are capable of producing at least 1 pg/cell/day, at least 2 pg/cell/day, at least 3 pg/cell/day, at least 4 pg/cell/day, at least 5 pg/cell/day, at least 6 pg/cell/day, at least 7 pg/cell/day, at least 8 pg/cell/day, at least 9 pg/cell/day, at least 10 pg/cell/day, at least 11 pg/cell/day, at least 12 pg/cell/day, at least 13 pg/cell/day, at least 14 pg/cell/day, at least 15 pg/cell/day, at least 20 pg/cell/day, at least 25 pg/cell/day, or more, preferably up to 50 pg/cell/day in a 10-day culture grown in fed batch or perfusion conditions, using a feed medium suitable for the mammalian host cell and containing amino acids, vitamins, or trace elements. In specific embodiments, mammalian host cells of the present disclosure express an protein of interest and have a specific productivity of at least 10 pg/cell/day, at least 11 pg/cell/day, at least 12 pg/cell/day, at least 13 pg/cell/day, at least 14 pg/cell/day, at least 15 pg/cell/day, at least 20 pg/cell/day, at least 25 pg/cell/day, or more, preferably up to 50 pg/cell/day under the culture conditions described above.

The mammalian host cells described herein can be used to express a protein of interest. The expressed protein may be secreted into the culture medium from which they can be recovered and/or collected. In addition, the proteins can be purified, or partially purified, from such culture or component (e.g., from culture medium) using known processes and products available from commercial vendors. The purified proteins can then be “formulated”, meaning buffer exchanged, sterilized, bulk-packaged, and/or packaged for a final user. Suitable formulations for pharmaceutical compositions include those described in Remington's Pharmaceutical Sciences, 18th ed. 1995, Mack Publishing Company, Easton, Pa.

A variety of known techniques can be utilized in making the polynucleotides, polypeptides, vectors, host cells, immune cells, compositions, and the like according to the invention.

The present invention is not to be limited in scope by the specific embodiments described herein that are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims.

### EXAMPLES

### Materials and Methods

### Plasmid Transfection and Pool Recovery

20 μg of a proprietary plasmid containing the gene of interest (GOI) and 5 μg of ILT piggyback transposase (see International Patent Application Publication No. WO2020123327) was transfected with a GenePulser Xcell™ electroporator (Bio-Rad Laboratories, Inc., Hercules, CA) into 20×10⁶ Glutamine Synthetase Knock Out (GSKO) Chinese Hamster Ovary (CHO) cells suspended in SAFC™ PF-CHO medium (Ex-Cell® 325 PF-CHO Serum-Free Medium, Cat. No. 14340C, Millipore Sigma, St. Louis, MO). 20 ml of chemically defined host cell medium was used to culture the transfected CHO cells immediate after transfection. Chemically defined glutamine-negative medium supplemented with 12.5 μM or 25 μM of L-Methionine sulfoximine (MSX, Millipore Sigma, St. Louis, MO) was used as selection medium 3 days after transfection. Transfected pools were passaged every 3 or 4 days until viability was 85% or higher. Viability and cell density were measured with Vi-CELL™ BLU cell viability analyzer (Beckman Coulter, Indianapolis, IN). Once viability was >85%, the cultures were seeded at 5×10⁵ cells/mL for 2 additional passages until viability and doubling time were stable.

### Fed-Batch

A 15-day Fed-Batch (FB) cell culture process was used to evaluate and rank the pools under production conditions. N−1 cultures were seeded at 5×10⁵ cells/ml in 10 ml glutamine-negative host medium without MSX. 4 days later, production cultures were seeded at 1×10⁶ cells/ml in 10 ml of glutamine-negative culture medium plus 1:750 Gibco™ Anti-Clumping Agent (Thermo Fisher Scientific Inc., Waltham, MA) and without MSX. The production cultures were carried up to day 15 as long as the viability was above 50%. Cultures were counted on days 3, 6, 8, 10, 13 and 15 using Vi-CELL™ BLU (Beckman Coulter Inc.) and the viable cell count and percent viability were recorded. Glucose levels in the media were measured on days 3, 6, 8, 10 and 13 using BioProfile® Flex2 Automated Cell Culture Analyzer (Nova Biomedical, Waltham, MA). Production cultures were fed with 5% v/v a proprietary media supplemented with 0.2% v/v tyrosine-cystine and glucose on days 3, 6, 8, 10 and 13. After glucose measurement, glucose levels were adjusted to 12-14 g/L depending on the glucose consumption with a 50% glucose stock solution on days 3, 6, 8, 10 and 13. 200 μl of harvest cell culture fluid (HCCF) was submitted for titer measurement on days 10, 13 and 15 to evaluate productivity.

On day 15, cell culture was spun down at 2000 rpm for 10 minutes and HCCF was submitted for ATOLL (a Protein A-HPLC), Size Exclusion Chromatography (SEC), reduced Capillary Electrophoresis (rCE) and non-reduced Capillary Electrophoresis (nrCE) to evaluate product quality. Titer and product quality data were visualized with TIBCO Spotfire® software (Cloud Software Group, Inc.). Only data from cultures with >60% viability were used for analysis. Values on the graphs represent the average of two replicate cultures. Fold change was calculated for the 12.5 μM MSX condition and the 25 μM MSX condition respectively. The fold change presented on the graph was the average fold change between the 12.5 UM and 25 μM MSX conditions.

### Example 1

As part of our vector screening effort to improve the productivity and product quality of 3- and 4-chain molecules, including a 3-chain molecule with a heavy chain fused to a cytokine and a heteroIgG 4-chain molecule, in a GSKO host, were tested with various combinations of promoters.

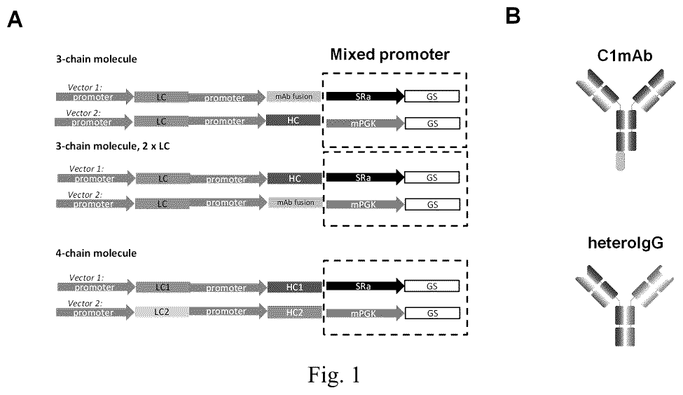

Two-vector systems were employed where different combinations of mPGK and Srα promoters were used to drive expression of glutamine synthetase as a selectable marker. In this experiment, a 3-chain molecule with a heavy chain fused to a cytokine was produced where the first vector encoded a light chain and a heavy chain and the second vector encoded a light chain and a heavy chain-peptide fusion. FIG. 1 shows a schematic of the different vectors used in this experiment.

Vectors were co-transfected into CHO cells and the cells cultures as described above. Titers and product quality were determined as described above.

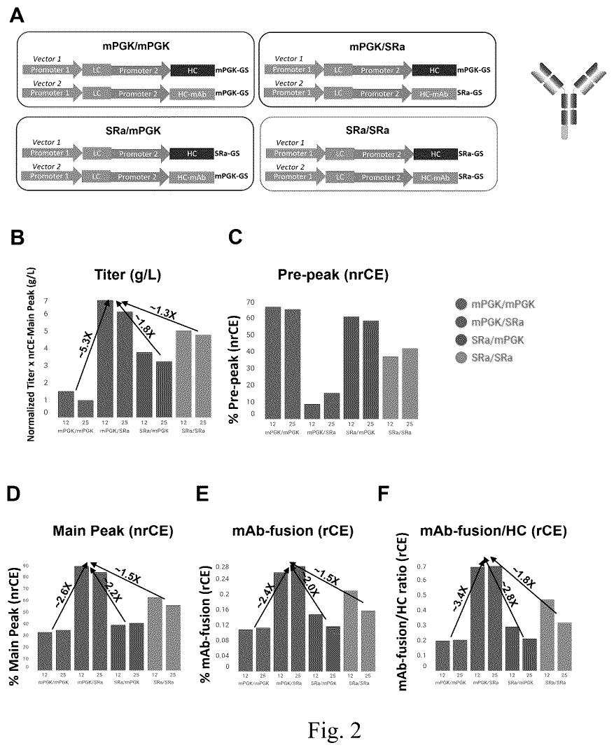

Pools using the mPGK promoter for the GS selection marker on one vector and the Srα promoter for the other vector had higher titers and product quality compared to pools where mPGK or Srα promoter was used to drive GS expression in both vectors. See FIG. 2. Improvements in titer and product quality were most evident when Srα was on the vector containing the expression-limiting chain (more difficult-to-express chain). In the case of the 3-chain molecule, up to 5.3-fold increase in effective titer (nrCE) and up to 2.6-fold improved product quality (nrCE % main peak) was observed for mPGK/Srα pools in comparison to mPGK/mPGK and Srα/Srα pools (FIG. 2). In particular, the mixed vector combination, mPGK/Srα, containing Srα on the vector with the more difficult-to-express mAb-fusion helped to increase expression of mAb-fusion up to 2.4-fold resulting in an increased ratio of mAb-fusion to heavy chain ratio, more balanced chain expression, and improved product quality.

### Example 2

As part of our vector screening effort to improve the productivity and product quality of 3- and 4-chain molecules, including a 3-chain molecule with a heavy chain fused to a cytokine and a heteroIgG 4-chain molecule, in a GSKO host, were tested with various combinations of promoters.

Two-vector systems were employed where different combinations of mPGK and Srα promoters were used to drive expression of glutamine synthetase as a selectable marker. In this experiment, a heteroIgG was produced where the first vector encoded a light chain and a heavy chain, and the second vector encoded a light chain and a heavy chain-protein fusion.

25 μM MSX was used during cell growth to favor selection of the vector containing Srα-GS and expression of the more difficult to express chain. Srα is a stronger promoter relative to mPGK so selection by Srα vector would be easier to overcome than selection of the mPGK vector at the higher 25 μM MSX concentration. By putting the difficult-to-express chain on the less stringent vector and the easier-to-express chain on the harsher selection, chain expression can be balanced.

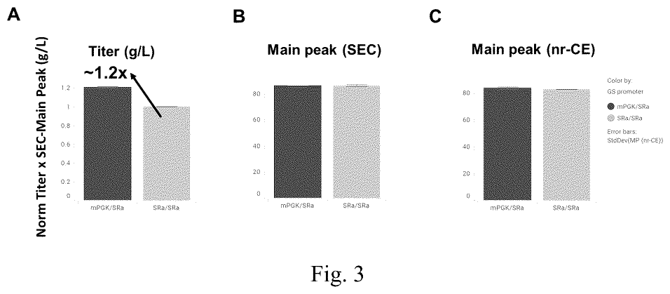

For heteroIgG, it was also observed that co-transfection of vectors with two different GS selection promoters (mPGK for the easier-to-express LC-HC and Srα for more difficult-to-express LC-HC) led to a 1.2-fold improvement in titer was observed in the corresponding Srα/Srα vector with minimal impact on product quality (FIG. 3).

Overall, these results suggest that the promoter for selection marker expression can also be used as a vector design lever for producing high-quality pools for similar 3- and 4-chain molecules, help to improve process efficiency, and reduce future costs for manufacturing.

What is claimed is:

1. A mammalian host cell for expressing an antigen binding protein having three or four different chains comprising a first expression vector and a second expression vector, wherein:
(a) the first expression vector comprises nucleotide sequences encoding 1) a first chain and a second chain, wherein the first chain is operably linked to a first promoter, the second chain is operably linked to a second promoter or an IRES sequence or is linked to the first chain through a linker sequence; and 2) a first selectable marker operably linked to a third promoter; and
(b) the second expression vector comprises nucleotide sequences encoding 1) a third chain and a fourth chain, wherein the third chain is operably linked to a fourth promoter, the fourth chain is operably linked to a fifth promoter or an IRES sequence or is linked to the third chain through a linker sequence; and 2) a second selectable marker operably linked to a sixth promoter,
wherein the first, second, third and fourth chains are selected from the group consisting of a heavy chain, a light chain, an antibody heavy chain fusion, an antibody light chain fusion, an ScFv, and an ScFv-Fc, wherein two of the chains may be identical; and
wherein the sixth promoter is different from the third promoter.

2. The mammalian host cell of claim 1, wherein the second chain and the fourth chain are operably linked to a second promoter and fourth promoter, respectively.

3. The mammalian host cell of claim 1, wherein the first selectable marker and the second selectable marker are the same.

4. The mammalian host cell of claim 3, wherein the first selectable marker and the second selectable marker are selected from the group consisting of glutamine synthetase and dihydrofolate reductase.

5. The mammalian host cell of claim 4, wherein the first selectable marker and the second selectable marker are glutamine synthetase.

6. The mammalian host cell of claim 1, wherein the third promoter and the sixth promoter are selected from mPGK and Srα.

7. The mammalian host cell of claim 2, wherein the first, second, fourth and fifth promoters are different from the third and sixth promoters.

8. The mammalian host cell of claim 7, wherein the first, second, fourth and fifth promoters are selected from the group consisting of CMV/GAPDH, CMV/adL, and CMV/EF1α.

9. The mammalian host cell of claim 7, wherein the first and fourth promoters are identical and the second and fifth promoters are identical.

10. The mammalian host cell of claim 9, wherein the first, second, fourth and fifth promoters are identical.

11. The mammalian host cell of claim 9, wherein the first and fourth promoters are different from the second and fifth promoters.

12. The mammalian host cell of claim 1, wherein the stronger promoter of the third promoter and the sixth promoter is on the expression vector with the more difficult-to-express chains.

13. The mammalian host cell of claim 12, wherein Srα is on the expression vector with the more difficult-to-express chains and mPGK is on the expression vector with the easier-to-express chains.

14. The mammalian host cell of claim 5, wherein methionine sulfoximine (MSX) stringency is optimized to favor expression of the more difficult-to-express chain paired with the stronger promoter.

15. The mammalian host cell of claim 14, wherein the MSX stringency is less than 75 μM.

16. The mammalian host cell of claim 1, wherein the stronger promoter of the third promoter and the sixth promoter is on the expression vector with the easier-to-express chains.

17. The mammalian host cell of claim 1, wherein the first and third chains are antibody light chains and the second and fourth chains are antibody heavy chains.

18. The mammalian host cell of claim 17, wherein the first and second chains are antibody light chains that are identical.

19. The mammalian host cell of claim 17, wherein the first and second chains are antibody light chains that are different.

20. The mammalian host cell of claim 1, wherein a) the first and third chains are antibody light chains and b) one of the second or fourth chain is an antibody heavy chain and the other is an antibody heavy chain fusion.

21. The mammalian host cell of claim 20, wherein the first and second chains are antibody light chains that are identical.

22. The mammalian host cell of claim 20, wherein the first and second chains are antibody light chains that are different.

23. The mammalian host cell of claim 1, wherein the antigen binding protein having three or four chains is selected from a heteroIgG, and a C1mAb.

24. The mammalian host cell of claim 1, wherein the first expression vector comprises in 5′ to 3′ order a first promoter, a nucleotide sequence encoding a first antibody light chain, a second promoter or an IRES, a nucleotide sequence encoding a first heavy chain, a third promoter which is Srα, and a nucleotide sequence encoding a selectable marker which is glutamine synthetase; and the second expression vector comprises in 5′ to 3′ order a fourth promoter, a nucleotide sequence encoding a second antibody light chain, a fifth promoter or an IRES, a nucleotide sequence encoding a second heavy chain, a sixth promoter which is mPGK, and a nucleotide sequence encoding a selectable marker which is glutamine synthetase.

25. The mammalian host cell of claim 24, wherein the first antibody light chain and the second antibody light chain are identical.

26. The mammalian host cell of claim 24, wherein the first heavy chain is more difficult-to-express than the second heavy chain.

27. The mammalian host cell of claim 1, wherein the first expression vector comprises in 5′ to 3′ order a first promoter, a nucleotide sequence encoding a first antibody light chain, a second promoter, a nucleotide sequence encoding a first heavy chain, a third promoter which is Srα, and a nucleotide sequence encoding a selectable marker which is glutamine synthetase; and the second expression vector comprises in 5′ to 3′ order a fourth promoter, a nucleotide sequence encoding a second antibody light chain, a fifth promoter, a nucleotide sequence encoding a second heavy chain, a sixth promoter which is mPGK, and a nucleotide sequence encoding a selectable marker which is glutamine synthetase.

28. The mammalian host cell of claim 27, wherein the first antibody light chain and the second antibody light chain are identical.

29. The mammalian host cell of claim 27, wherein the first heavy chain is more difficult-to-express than the second heavy chain.

30. The mammalian host cell of claim 1, wherein the first expression vector comprises in 5′ to 3′ order a first promoter, a nucleotide sequence encoding an antibody light chain, a second promoter or an IRES, a nucleotide sequence encoding a heavy chain fusion, a third promoter which is Srα, and a nucleotide sequence encoding a selectable marker which is glutamine synthetase; and the second expression vector comprises in 5′ to 3′ order a fourth promoter, a nucleotide sequence encoding the antibody light chain, a fifth promoter or an IRES, a nucleotide sequence encoding a heavy chain, a sixth promoter which is mPGK, and a nucleotide sequence encoding a selectable marker which is glutamine synthetase.

31. The mammalian host cell of claim 30, wherein the first antibody light chain and the second antibody light chain are identical.

32. The mammalian host cell of claim 1, wherein the first expression vector comprises in 5′ to 3′ order a first promoter, a nucleotide sequence encoding an antibody light chain, a second promoter, a nucleotide sequence encoding a heavy chain fusion, a third promoter which is Srα, and a selectable marker which is glutamine synthetase; and the second expression vector comprises in 5′ to 3′ order a fourth promoter, a nucleotide sequence encoding the antibody light chain, a fifth promoter, a nucleotide sequence encoding a heavy chain, a sixth promoter which is mPGK, and a nucleotide sequence encoding a selectable marker which is glutamine synthetase.

33. The mammalian host cell of claim 32, wherein the first antibody light chain and the second antibody light chain are identical.

34. The mammalian host cell of claim 1, wherein the first and second expression vectors are integrated in the genome of the host cell.

35. The mammalian host cell of claim 1, wherein the host cell is a Chinese Hamster Ovary (CHO) cell.

36. The mammalian host cell of claim 35, wherein the CHO cell is deficient in dihydrofolate reductase (DHFR) or is a glutamine synthetase knock out (GSKO).

37. A method for producing an antigen binding protein having three or four chains comprising
a) culturing the mammalian host cell of claim 1 under conditions to express the antigen binding protein; and
b) recovering the antigen binding protein.

38. The method of claim 37, wherein the recovered antigen binding protein is purified and formulated in a pharmaceutically acceptable formulation.

39. A method for producing an antigen binding protein having three or four chains comprising
a) culturing the mammalian host cell of claim 36 under conditions to express the antigen binding protein and under methotrexate stringency in the case of a CHO DHFR-cell or methionine sulfoxamine stringency in the case of a CHO GSKO cell to favor expression of the difficult-to-express chain paired with the stronger GS promoter; and
b) recovering the antigen binding protein.

40. The method of claim 39, wherein the recovered antigen binding protein is purified and formulated in a pharmaceutically acceptable formulation.
